(19) United States
(12) Patent Application Publication
(10) Pub. No.: US 2026/0286378 A1
(43) Pub. Date: Sep. 24, 2026

(54) GENE EDITED MAIZE WITH ALTERED CHARACTERISTICS

(71) Applicants: VIB vzw, Gent (BE); Universiteit Gent, Gent (BE); INARI AGRICULTURE TECHNOLOGY, INC., Cambridge, MA (US); Instituut Voor Landbouw-En Visserijonderzoek (ILVO), Merelbeke (BE)

(72) Inventors: Tom Van Hautegem, Brakel (BE); Hannes Bart Claeys, Gent (BE); Frédéric Van Ex, Kortrijk (BE); Michael Lee Nuccio, Salem, NH (US); Hilde Nelissen, Ledeberg (BE); Dirk Inzé, Aalst (BE); Tom Ruttink, Gent (BE)

(21) Appl. No.: 19/475,818

(22) PCT Filed: Apr. 17, 2024
(86) PCT No.: PCT/US2024/024962

§ 371 Date: Oct. 16, 2025

Related U.S. Application Data
(60) Provisional application No. 63/496,774, filed on Apr. 18, 2023.

(51) Int. Cl. C12N 15/82; C12N 9/22; C12N 15/11; C12Q 1/6895

(52) U.S. Cl. CPC C12N 15/8273; C12N 9/226; C12N 15/111; C12N 15/8201; C12N 15/8274; C12N 15/8279; C12Q 1/6895; C12N 2310/20; C12Q 2600/13

(57) ABSTRACT

The disclosure relates to maize plants with altered characteristics including tolerance to abiotic stress such as drought and/or increased water use efficiency, along with methods of using and making the same.

### CROSS REFERENCE TO RELATED APPLICATIONS

This application claims priority to provisional application U.S. Ser. No. 63/496,774, filed Apr. 18, 2023, which is hereby incorporated herein by reference in its entirety.

### SEQUENCE LISTING XML

The instant application contains a sequence listing, which has been submitted in XML file format by electronic submission and is hereby incorporated by reference in its entirety. The XML file, created on Apr. 17, 2024, is named P14339W000.xml and is 47,783 bytes in size.

### TECHNICAL FIELD

The present disclosure relates to maize plants having tolerance to abiotic stress (e.g., drought) as well as compositions and methods for increasing abiotic stress (e.g., drought) tolerance in maize.

### BACKGROUND

Agriculture is an essential industry for the global economy and the United States in particular. Drought is one of the most devastating causes of yield losses in maize and the prospect of climate change-driven increases in severity and duration of drought spells poses an imminent threat to agricultural productivity.

### SUMMARY

Disclosed herein are maize plants comprising a loss-of-function allele of the endogenous maize gene of SEQ ID NO: 1, 3, 5, 7, 9, and/or 11, or an allelic variant thereof. Also provided are maize plant parts and maize plant cells of the aforementioned maize plants.

Methods of producing maize seed comprising growing the aforementioned maize plants and harvesting seed therefrom are provided. Biological samples comprising a nucleic acid containing a loss-of-function allele of the endogenous maize gene of SEQ ID NO: 1, 3, 5, 7, 9, and/or 11, or an allelic variant thereof are provided. Also provided are methods of producing a commodity maize plant product comprising processing the aforementioned maize plants or a maize seed obtained therefrom; and recovering the commodity maize plant product from the processed maize plant or maize seed.

Methods for generating the aforementioned maize plants comprising introducing a loss-of-function allele in the endogenous maize gene of SEQ ID NO: 1, 3, 5, 7, 9, and/or 11, or an allelic variant thereof are provided. Guide RNA molecules comprising a spacer RNA molecule that targets the endogenous maize gene of SEQ ID NO: 1, 3, 5, 7, 9, 11, or an allelic variant thereof are provided.

Methods for determining whether a maize plant cell, plant part, plant, or biological sample obtained therefrom comprises a loss-of-function allele of the endogenous maize gene of SEQ ID NO: 1, 3, 5, 7, 9, and/or 11, or an allelic variant thereof are provided. In certain embodiments, the methods comprise analyzing a polynucleotide comprising a portion of SEQ ID NO: 1, 3, 5, 7, 9, and/or 11, or an allelic variant thereof, or analyzing an RNA encoded by a portion of SEQ ID NO: 1, 3, 5, 7, 9, and/or 11, or an allelic variant thereof, from the maize plant cell, plant part, plant, or biological sample, wherein an insertion, deletion, and/or substitution of one or more nucleotides in the polynucleotide or RNA is indicative of the presence of the loss-of-function allele in the maize plant cell, plant part, plant, or biological sample. In certain embodiments, the method comprise analyzing a polypeptide encoded by SEQ ID NO: 1, 3, 5, 7, 9, and/or 11, a portion thereof, or an allelic variant thereof from the maize plant cell, plant part, or plant, wherein an insertion, deletion, and/or substitution of one or more amino acid residues of the polypeptide or a change in the biologic or biochemical activity of the polypeptide is indicative of the presence of the loss-of-function allele in the maize plant cell, plant part, plant, or biological sample.

### DESCRIPTION OF THE DRAWINGS

FIG. 1 shows the Pearson correlation between LER and LED in different environments. Scatter plot of LER and LED in well-watered (WW) and water deficit (WD) conditions for the 23 selected RILs and the two parental lines (n=8 for all conditions, except for B73 and H99 n=27, for RIL109 and RIL116 n=16). LER and LED are not significantly correlated (P=0.293) in well-watered conditions and significantly negatively correlated (P=0.001) by water deficit. Statistical significance was determined by Student's t-test. All measurements were performed on the fourth leaf.

FIG. 2A-C shows detailed analysis of the effect of rehydration. Leaf elongation rates of RIL89 in well-watered (WW), water deficit (WD) and rehydration (RW) conditions. Water deficit plants were rehydrated to well-watered conditions one (FIG. 2A), four (FIG. 2B) and six (FIG. 2C) days after leaf four appearance (DALA). Error bars indicate the standard error of the mean (n=50 for WW; 35 for WD; 17 for RW1; 16 for RW4 and 17 for RW6). Time of RW is indicated with an arrow.

FIG. 3A-C shows that growth recovery after rehydration depends on cell division activity. FIG. 3A shows the size of the division zone (DZ) of RIL89 plants grown in water deficit (WD) conditions at different days after leaf appearance (DALA). Error bars indicate the standard error of the mean. Statistical significance was determined by Student's t-test (*** P<0.001; ** P<0.01 and *P<0.05; n=4 for 2, 5 and 7 days after leaf appearance; n=3 for 3, 4, 6 and 8 days after leaf appearance). All measurements were performed on the fourth leaf FIG. 3B shows the size of the division zone (DZ) of RIL89 two, five and seven days after leaf appearance (DALA) in well-watered (WW), water deficit (WD) and rehydration (RW) conditions. Statistical significance was determined by two-way ANOVA followed by Tukey HSD (***P<0.001). Error bars indicate the standard error of the mean (n=4 for all conditions, except for well-watered and water deficit at 7 days after leaf appearance n=3). All measurements were performed on the fourth leaf. FIG. 3C shows the experimental setup for the RNA-sequencing experiment of the leaf four samples (basal half cm indicated by the dashed line) of RIL 89 plants. Rehydrated samples were harvested at five and seven days after leaf appearance. Well-watered and water deficit samples were harvested four, five, six and seven days after leaf appearance. For each condition, three biological replicates of three plants each were harvested. The division zone is indicated with hatching lines.

FIG. 4A shows division zone size measurements in drought-treated GRF1 transgenic (T) and non-transgenic (NT) plants at different days after leaf appearance (DALA). Error bars indicate the standard error of the mean (n=4 for all conditions, except for GRF1 NT_3DALA n=5 and for GRF1 T_3DALA, GRF1 T_7DALA n=3). Statistical significance was determined by two-way ANOVA statistical test with Tukey HSD (***P<0.001). All measurements were performed on the fourth leaf. FIG. 4B-G shows leaf elongation rates (LER) of GRF1 transgenic (T) and non-transgenic (NT) plants in well-watered (WW), water deficit (WD) and rehydration (RW) conditions. Water deficit plants were rehydrated to well-watered conditions two (FIG. 4B-C), five (FIG. 4D-E) and six (FIG. 4F-G) days after leaf appearance (DALA). Error bars indicate the standard error of the mean (n=11 for WW_NT; 5 for WW_T; 9 for WD_NT; 6 for WD_T; 5 for RW2_NT; 5 for RW2_T; 3 for RW5_NT; 4 for RW5_T; 5 for RW6_NT and 4 for RW6_T). Time of rehydration is indicated with an arrow. All measurements were performed on the fourth leaf.

FIG. 5 shows the effect on final leaf length for gene-edited populations under water deficit (WD) conditions. Measurements of final leaf length 3 (FLL3) for two populations of Script Drought 1 (SD1A and B), Script Drought 2 (SD2A and B), Script Drought 3 (SD3A and B), Script Drought 4 (SD4A and B) and Script Drought 5 (SD5A and B) compared to the EDITOR 1 (ED1) background controls. Each dot represents one individual per population and bars represent the mean+SEM. A total of at least 45 plants were assayed for each SD population and 20 plants for ED1.

FIG. 6A-C shows pictures of general plant architecture (FIG. 6A) as well as lateral (FIG. 6B) and front (FIG. 6C) view of leaves from different plants from ED1 and SD3A as examples.

FIG. 7 shows network showing an association analysis of single-gene loss of function (LOF) and important agronomical traits. Single-gene associations were performed per population. Results are summarized with two indices. 1) P-value: the significance of the respective connections between genes and traits is proportional to the line width. 2) Line color represents the largest difference to group LOF0/2: percentage difference for a specific trait between siblings that have LOF2/2 to individuals that do not have mutations at that locus (LOF0/2). Associations displaying a large difference to LOF0/2, either positive or negative, along with a low P-value (thick line) indicate strong evidence for a gene effect on the trait. Traits are displayed in bold. LDW, leaf dry weight; SFW, stem fresh weight; SDW, stem dry weight; ASI, anthesis-silking interval; PEW, primary ear weight; PEL, primary ear length.

FIG. 8 shows association analysis of single-gene loss of function (LOF) and traits. Single-gene associations were performed per population. Results are summarized per gene and per trait with two indices. 1) log 10pval: the significance of the respective association. 2) Largest difference to group LOF0/2: percentage difference for a specific trait between siblings that have LOF2/2 to individuals that do not have mutations at that locus (LOF0/2). Associations displaying a large difference to LOF0/2, either positive or negative, along with a low P-value indicate strong evidence for a gene effect on the trait.

FIG. 9A-I shows distributions for different traits evaluated under water deficit for REW genes in the multiplex gene-edited populations. Lamina length of leaf 7 (FIG. 9A) and leaf 8 (FIG. 9B) and leaf dry weight (FIG. 9C) for SD2 plants edited in ZmGRF12. FIG. 9D-F shows lamina length of leaf 14 (FIG. 9D), leaf dry weight (FIG. 9E) and days to anthesis (FIG. 9F) for SD3 plants edited in ZmZHD1. FIG. 9G-I shows stem fresh weight (FIG. 9G), stem dry weight (FIG. 911) and leaf dry weight (FIG. 91) for SD5 plants edited in ZmbZIP100. On the X-axis the percentage of editing is depicted: LOF_{0/2}=wild-type; LOF_{1/2}: heterozygous; LOF_{2/2} homozygous/biallelic. On the distributions, each dot represents one individual; the middle line of box plot represents the mean and the whiskers represent the SD.

### DETAILED DESCRIPTION

The phrase “allelic variant” as used herein refers to a polynucleotide or polypeptide sequence variant that occurs in a particular gene at particular locus in a different strain, variety, or isolate of a given organism.

As used herein, the phrase “amorphic allele” refers to an allele of a gene having no gene activity in comparison to the wild-type allele of the gene. Amorphic alleles are also known as null alleles.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “biological sample” refers to either intact or non-intact (e.g., milled seed or plant tissue, chopped plant tissue, lyophilized tissue) plant tissue. It may also be an extract comprising intact or non-intact seed or plant tissue. The biological sample can comprise flour, meal, syrup, oil, starch, and cereals manufactured in whole or in part to contain crop plant by-products. In certain embodiments, the biological sample is “non-regenerable” (i.e., incapable of being regenerated into a plant or plant part).

As used herein, the terms “correspond,” “corresponding,” and the like, when used in the context of an nucleotide position, mutation, and/or substitution in any given polynucleotide (e.g., an allelic variant of SEQ ID NO: 1) with respect to the reference polynucleotide sequence (e.g., SEQ ID NO: 1) all refer to the position of the nucleotide in the given sequence that has identity to the nucleotide in the reference nucleotide sequence when the given polynucleotide is aligned to the reference polynucleotide sequence using a pairwise alignment algorithm (e.g., CLUSTAL O 1.2.4 with default parameters).

As used herein, the terms “Cpf1” and “Cas12a” are used interchangeably to refer to the same RNA dependent DNA endonuclease (RdDe).

As used herein “drought conditions” refers to a period of dryness (acute or chronic/prolonged) that results in water deficit and subjects plants to stress and/or damage to plant tissues and/or negatively affects grain/crop yield.

As used herein, the phrase “elite crop plant” refers to a plant which has undergone breeding to provide one or more trait improvements. Elite crop plant lines include plants which are an essentially homozygous, e.g., inbred or doubled haploid. Elite crop plants can include inbred lines used as is or used as pollen donors or pollen recipients in hybrid seed production (e.g., used to produce F1 plants). Elite crop plants can include inbred lines which are selfed to produce non-hybrid cultivars or varieties or to produce (e.g., bulk up) pollen donor or recipient lines for hybrid seed production. Elite crop plants can include hybrid F1 progeny of a cross between two distinct elite inbred or doubled haploid plant lines.

As used herein, the phrase “endogenous gene” refers to the native form of a gene unit in its natural location in the genome of an organism.

As used herein, the term “expression” refers to the production of a functional end-product (e.g., an mRNA, guide RNA, or a protein) in either precursor or mature form.

As used herein, the phrase “hypomorphic allele” refers to an allele of a gene with less gene activity than a wild-type allele but more gene activity than an amorphic allele.

As used herein, the terms “include,” “includes,” and “including” are to be construed as at least having the features to which they refer while not excluding any additional unspecified features.

As used herein, the term “isomorphic allele” refers to an allele of a gene having wild-type gene activity.

The term “isolated” as used herein means having been removed from its natural environment.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, a “loss-of-function allele” can include an amorphic allele or a hypomorphic allele of a gene.

The phrase “marker-assisted selection”, as used herein, refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The phrase “molecular marker”, as used herein, refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, Next Generation Sequencing (NGS) of a molecular marker, cleaved amplified polymorphic sequence (CAPS) markers or isozyme markers or combinations of the markers described herein which defines a specific genetic and chromosomal location.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

The terms “selfed,” “selfing,” and “self,” as used herein, refer to any process used to obtain progeny from the same plant or plant line as well as to plants resulting from the process.

As used herein, the terms thus include any fertilization process wherein both the ovule and pollen are from the same plant or plant line and plants resulting therefrom. Typically, the terms refer to self-pollination processes and progeny plants resulting from self-pollination.

The term “selecting”, as used herein, refers to a process of picking out a certain individual plant from a group of individuals, usually based on a certain identity, trait, characteristic, and/or molecular marker of that individual.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The present disclosure provides for maize plant cells, plant parts including seed, plants, and biological samples comprising a loss-of-function allele of the endogenous maize gene of SEQ ID NO: 1, 3, 5, 7, 9, and/or 11, or an allelic variant thereof. These maize plants and parts can be utilized for human food, livestock feed, as a raw material in industry, or as breeding material for development of other maize plants.

In certain embodiments, the target endogenous maize gene comprises Zm00001eb378820. The endogenous maize Zm00001eb378820 gene comprises the genomic DNA of SEQ ID NO: 1 and allelic variants thereof located on maize chromosome 9. The endogenous maize Zm00001eb378820 gene is located at nucleotides 27,726,973 to 27,729,989 of chromosome 9 of the Zea mays B73 genome assembly version 5 (Zm-B73-REFERENCE-NAM-5.0 on the world wide web internet site “maizegdb.org”). The endogenous maize Zm00001eb378820 gene has also been referred to as Zm00001d045533 (Zm-B73-REFERENCE-GRAMENE-4.0), GRMZM2G119359 (B73 RefGen v3), and LOC100125642 (NCBI). Allelic variants of an endogenous maize Zm00001eb378820 gene include variants which encode proteins having at least 95%, 96%, 97%, 98%, 99%, 99.5%, 99.6%, 99.7%, 99.8%, or 99.9% sequence identity to SEQ ID NO 2. Allelic variants of an endogenous maize Zm00001eb378820 gene also include variants which comprise genomic DNA having at least 95%, 96%, 97%, 98%, 99%, 99.5%, 99.6%, 99.7%, 99.8%, or 99.9% sequence identity to SEQ ID NO: 1. In certain embodiments, allelic variants of the endogenous maize Zm00001eb378820 gene are isomorphic alleles of the endogenous maize Zm00001eb378820 gene. The wild-type maize Zm00001eb378820 gene encodes a GRF transcription factor.

In certain embodiments, the target endogenous maize gene comprises Zm00001eb398520. The endogenous maize Zm00001eb398520 gene comprises the genomic DNA of SEQ ID NO: 3 and allelic variants thereof located on maize chromosome 9. The endogenous maize Zm00001eb398520 gene is located at nucleotides 148,417,192 to 148,422,766 of chromosome 9 of the Zea mays B73 genome assembly version 5 (Zm-B73-REFERENCE-NAM-5.0). The endogenous maize Zm00001eb398520 gene has also been referred to as Zm00001d047909 (Zm-B73-REFERENCE-GRAMENE-4.0), GRMZM2G145085 (B73 RefGen_v3), and LOC103639350 (NCBI). Allelic variants of an endogenous maize Zm00001eb398520 gene include variants which encode proteins having at least 95%, 96%, 97%, 98%, 99%, 99.5%, 99.6%, 99.7%, 99.8%, or 99.9% sequence identity to SEQ ID NO: 4. Allelic variants of an endogenous maize Zm00001eb398520 gene also include variants which comprise genomic DNA having at least 95%, 96%, 97%, 98%, 99%, 99.5%, 99.6%, 99.7%, 99.8%, or 99.9% sequence identity to SEQ ID NO: 3. In certain embodiments, allelic variants of the endogenous maize Zm00001eb398520 gene are isomorphic alleles of the endogenous maize Zm00001eb398520 gene. The wild-type maize Zm00001eb398520 gene encodes a transcription initiation factor IIF subunit alpha.

In certain embodiments, the target endogenous maize gene comprises Zm00001eb168120. The endogenous maize Zm00001eb168120 gene comprises the genomic DNA of SEQ ID NO: 5 and allelic variants thereof located on maize chromosome 4. The endogenous maize Zm00001eb168120 gene is located at nucleotides 12,317,574-12,320,379 of chromosome 4 of the Zea mays B73 genome assembly version 5 (Zm-B73-REFERENCE-NAM-5.0). The endogenous maize Zm00001eb168120 gene has also been referred to as Zm00001d049000 (Zm-B73-REFERENCE-GRAMENE-4.0), GRMZM2G068330 (B73 RefGen_v3), and LOC103652837 (NCBI). Allelic variants of an endogenous maize Zm00001eb168120 gene include variants which encode proteins having at least 95%, 96%, 97%, 98%, 99%, 99.5%, 99.6%, 99.7%, 99.8%, or 99.9% sequence identity to SEQ ID NO: 6. Allelic variants of an endogenous maize Zm00001eb168120 gene also include variants which comprise genomic DNA having at least 95%, 96%, 97%, 98%, 99%, 99.5%, 99.6%, 99.7%, 99.8%, or 99.9% sequence identity to SEQ ID NO: 5. In certain embodiments, allelic variants of the endogenous maize Zm00001eb168120 gene are isomorphic alleles of the endogenous maize Zm00001eb168120 gene. The wild-type maize Zm00001eb168120 gene encodes a zinc finger-homeodomain (ZF-HD) transcriptional factor.

In certain embodiments, the target endogenous maize gene comprises Zm000015eb01380. The endogenous maize Zm00001eb051380 gene comprises the genomic DNA of SEQ ID NO: 7 and allelic variants thereof located on maize chromosome 1. The endogenous maize Zm00001eb051380 gene is located at nucleotides 261,028,189-261,029,535 of chromosome 1 of the Zea mays B73 genome assembly version 5 (Zm-B73-REFERENCE-NAM-5.0). The endogenous maize Zm00001eb051380 gene has also been referred to as Zm00001d033325 (Zm-B73-REFERENCE-GRAMENE-4.0), GRMZM2G045678 (B73 RefGen_v3), and LOC100283109 (NCBI). Allelic variants of an endogenous maize Zm00001eb051380 gene include variants which encode proteins having at least 95%, 96%, 97%, 98%, 99%, 99.5%, 99.6%, 99.7%, 99.8%, or 99.9% sequence identity to SEQ ID NO: 8. Allelic variants of an endogenous maize Zm00001eb051380 gene also include variants which comprise genomic DNA having at least 95%, 96%, 97%, 98%, 99%, 99.5%, 99.6%, 99.7%, 99.8%, or 99.9% sequence identity to SEQ ID NO: 7. In certain embodiments, allelic variants of the endogenous maize Zm00001eb051380 gene are isomorphic alleles of the endogenous maize Zm00001eb051380 gene. The wild-type maize Zm00001eb051380 gene encodes a C2C2-Dof-transcription factor.

In certain embodiments, the target endogenous maize gene comprises Zm00001eb320950. The endogenous maize Zm00001eb320950 gene comprises the genomic DNA of SEQ ID NO: 9 and allelic variants thereof located on maize chromosome 7. The endogenous maize Zm00001eb320950 gene is located at nucleotides 153,524,147-153,529,720 of chromosome 7 of the Zea mays B73 genome assembly version 5 (Zm-B73-REFERENCE-NAM-5.0). The endogenous maize Zm00001eb320950 gene has also been referred to as Zm00001d021442 (Zm-B73-REFERENCE-GRAMENE-4.0), GRMZM2G053298 (B73 RefGen_v3), and LOC103633146 (NCBI). Allelic variants of an endogenous maize Zm00001eb320950 gene include variants which encode proteins having at least 95%, 96%, 97%, 98%, 99%, 99.5%, 99.6%, 99.7%, 99.8%, or 99.9% sequence identity to SEQ ID NO: 10. Allelic variants of an endogenous maize Zm00001eb320950 gene also include variants which comprise genomic DNA having at least 95%, 96%, 97%, 98%, 99%, 99.5%, 99.6%, 99.7%, 99.8%, or 99.9% sequence identity to SEQ ID NO: 9. In certain embodiments, allelic variants of the endogenous maize Zm00001eb320950 gene are isomorphic alleles of the endogenous maize Zm00001eb320950 gene. The wild-type maize Zm00001eb320950 gene encodes a NIN-like protein (NLP) transcription factor.

In certain embodiments, the target endogenous maize gene comprises Zm00001eb373300. The endogenous maize Zm00001eb373300 gene comprises the genomic DNA of SEQ ID NO: 11 and allelic variants thereof located on maize chromosome 9. The endogenous maize Zm00001eb373300 gene is located at nucleotides 9,473,425-9,483,808 of chromosome 9 of the Zea mays B73 genome assembly version 5 (Zm-B73-REFERENCE-NAM-5.0). The endogenous maize Zm00001eb373300 gene has also been referred to as Zm00001d044940 (Zm-B73-REFERENCE-GRAMENE-4.0), GRMZM2G033413 (B73 RefGen_v3), and LOC103637889 (NCBI). Allelic variants of an endogenous maize Zm00001eb373300 gene include variants which encode proteins having at least 95%, 96%, 97%, 98%, 99%, 99.5%, 99.6%, 99.7%, 99.8%, or 99.9% sequence identity to SEQ ID NO: 12. Allelic variants of an endogenous maize Zm00001eb373300 gene also include variants which comprise genomic DNA having at least 95%, 96%, 97%, 98%, 99%, 99.5%, 99.6%, 99.7%, 99.8%, or 99.9% sequence identity to SEQ ID NO: 11. In certain embodiments, allelic variants of the endogenous maize Zm00001eb373300 gene are isomorphic alleles of the endogenous maize Zm00001eb373300 gene. The wild-type maize Zm00001eb373300 gene encodes a bZIP transcription factor.

Maize plant cells, plant parts, and plants comprising a loss-of-function allele of the endogenous maize gene of SEQ ID NO: 1, 3, 5, 7, 9, and/or 11 or an allelic variant thereof are provided. Examples of loss-of-function alleles can include a deletion, an insertion, and/or a substitution of one or more nucleotides of the endogenous maize gene. The insertion, deletion, and/or substitution can be made anywhere in the gene including, for example, in the promoter region, an exon, an intron, and/or the untranslated regions (5′ UTR or 3′ UTR). In certain embodiments, the loss-of-function allele comprises a deletion, insertion, and/or substitution in the coding region of the gene. In certain embodiments, a loss-of-function allele of the gene can comprise a deletion of the entire coding region or any portion of the coding region required for biological activity. In certain embodiments, the loss-of-function allele comprises a deletion and/or an insertion at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, 300, 350, 400, 450, 500, 600, 700, 800, 900, or 1000 nucleotides in the endogenous maize gene.

In certain embodiments, the loss-of-function allele comprises a deletion, an insertion, and/or substitution that results in a frameshift mutation and/or a nonsense mutation in the coding region of the endogenous maize gene (e.g., SEQ ID NO: 1, 3, 5, 7, 9, and/or 11 or an allelic variant thereof). In certain embodiments, the loss-of-function allele of the endogenous maize gene can comprise a deletion of any number of nucleotides that are not divisible by 3 in an exon of the endogenous maize gene. In certain embodiments, the loss-of-function allele of the endogenous maize gene can comprise a deletion of 1, 2, 4, 5, 7, 8, 10, 11, 13, 14, 16, 17, 19, 20, 22, 23, 25, 26, 28, 29, 31, 32, 34, 35, 37, 38, 40, 41, 43, 44, 46, 47, 49, 50, 52, 53, 55, 56, 58, 59, 61, 62, 64, 65, 67, 68, 70, 71, 73, 74, 76, 77, 79, 80, 82, 83, 85, 86, 88, 89, 91, 92, 94, 95, 97, 98, 100, 101, 103, 104, 106, 107, 109, 110, 112, 113, 115, 116, 118, 119, 121, 122, 124, 125, 127, 128, 130, 131, 133, 134, 136, 137, 139, 140, 142, 143, 145, 146, 148, 149, 151, 152, 154, 155, 157, 158, 160, 161, 163, 164, 166, 167, 169, 170, 172, 173, 175, 176, 178, 179, 181, 182, 184, 185, 187, 188, 190, 191, 193, 194, 196, 197, 199, or 200 nucleotides of the endogenous maize gene and result in a frameshift mutation. In certain embodiments, the loss-of-function allele comprises an internal deletion that preserves the reading frame of the encoded protein while removing at least one, two, or three codons, thus resulting in a protein lacking at least one, two, or three amino acid residues. In certain embodiments, the loss-of-function allele of the endogenous maize gene can comprise a deletion of any number of nucleotides that are divisible by 3 in an exon of the endogenous maize gene. In certain embodiments, the loss-of-function allele of the endogenous maize gene can comprise a deletion of 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, or 201 nucleotides of the endogenous maize gene and preserves the reading frame.

In certain embodiments, the water use efficiency and/or drought tolerance of a maize plant comprising the loss-of-function allele of the endogenous maize gene (e.g., SEQ ID NO: 1, 3, 5, 7, 9, and/or 11 or an allelic variant thereof) is increased in comparison to the water use efficiency and/or drought tolerance of a wild-type control maize plant lacking the loss-of-function allele. As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to drought conditions. The physiological responses of plants to a deficit of water include leaf wilting, a reduction in leaf area, leaf abscission, and the stimulation of root growth by directing nutrients to the underground parts of the plants. Plants are more susceptible to drought during flowering and seed development (the reproductive stages). As used herein “increased water use efficiency” refers to the ability of a plant to grow, develop, or yield faster or better than normal when subjected to the same amount of available/applied water as under normal or standard conditions. As used herein “increased drought tolerance” refers to the ability of plants to grow, develop, or yield normally, or to grow, develop, or yield faster or better than normal when subjected to reduced amounts of available/applied water and/or under conditions of acute or chronic drought.

In certain embodiments, the internode length of a maize plant comprising the loss-of-function allele of the endogenous maize gene (e.g., SEQ ID NO: 1, 3, 5, 7, 9, and/or 11 or an allelic variant thereof) is decreased in comparison to the internode length of a wild-type control maize plant lacking the loss-of-function allele. In certain embodiments, the internode length is decreased by at least about 2%, 3%, 4%, 5%, 6%, 7%, 8%, 9%, 10%, 12%, 15%, 18%, 20%, 25%, 30%, 35%, 40%, 45%, or 50% in comparison to the internode length of the corresponding wild-type control maize plant lacking the loss-of-function allele. In certain embodiments, the leaf lamina length of a maize plant comprising the loss-of-function allele of the endogenous maize gene (e.g., SEQ ID NO: 1, 3, 5, 7, 9, and/or 11 or an allelic variant thereof) is decreased in comparison to the leaf lamina length of a wild-type control maize plant lacking the loss-of-function allele. In certain embodiments, the leaf lamina length is decreased by at least about 2%, 3%, 4%, 5%, 6%, 7%, 8%, 9%, 10%, 12%, 15%, 18%, 20%, 25%, 30%, 35%, 40%, 45%, or 50% in comparison to the leaf lamina length of the corresponding wild-type control maize plant lacking the loss-of-function allele. In certain embodiments, the leaf sheath length of a maize plant comprising the loss-of-function allele of the endogenous maize gene (e.g., SEQ ID NO: 1, 3, 5, 7, 9, and/or 11 or an allelic variant thereof) is decreased in comparison to the leaf sheath length of a wild-type control maize plant lacking the loss-of-function allele. In certain embodiments, the leaf sheath length is decreased by at least about 2%, 3%, 4%, 5%, 6%, 7%, 8%, 9%0, 1, 2%1, 5%1, 18%, 20%, 25%, 30%, 35%, 40%, 45%, or 50% in comparison to the leaf sheath length of the corresponding wild-type control maize plant lacking the loss-of-function allele. In certain embodiments, the leaf dry weight of a maize plant comprising the loss-of-function allele of the endogenous maize gene (e.g., SEQ ID NO: 1, 3, 5, 7, 9, and/or 11 or an allelic variant thereof) is decreased in comparison to the leaf dry weight of a wild-type control maize plant lacking the loss-of-function allele. In certain embodiments, the leaf dry weight is decreased by at least about 2%, 3%, 4%, 5%, 6%, 7%, 8%, 9%, 10%, 12%, 15%, 18%, 20%, 25%, 30%, 35%, 40%, 45%, or 50% in comparison to the leaf dry weight of the corresponding wild-type control maize plant lacking the loss-of-function allele. In certain embodiments, the leaf dry matter content of a maize plant comprising the loss-of-function allele of the endogenous maize gene (e.g., SEQ ID NO: 1, 3, 5, 7, 9, and/or 11 or an allelic variant thereof) is decreased in comparison to the leaf dry matter content of a wild-type control maize plant lacking the loss-of-function allele. In certain embodiments, the leaf dry matter content is decreased by at least about 2%, 3%, 4%, 5%, 6%, 7%, 8%, 9%, 10%, 12%, 15%, 18%, 20%, 25%, 30%, 35%, 40%, 45%, or 50% in comparison to the leaf dry matter content of the corresponding wild-type control maize plant lacking the loss-of-function allele. In certain embodiments, the primary ear length of a maize plant comprising the loss-of-function allele of the endogenous maize gene (e.g., SEQ ID NO: 1, 3, 5, 7, 9, and/or 11 or an allelic variant thereof) is decreased in comparison to the primary ear length of a wild-type control maize plant lacking the loss-of-function allele. In certain embodiments, the primary ear length is decreased by at least about 2%, 3%, 4%, 5%, 6%, 7%, 8%, 9%, 10%, 12%, 15%, 18%, 20%, 25%, 30%, 35%, 40%, 45%, or 50% in comparison to the primary ear length of the corresponding wild-type control maize plant lacking the loss-of-function allele.

In certain embodiments, the internode length, leaf lamina length, leaf sheath length, leaf dry weight, leaf dry matter content, and/or primary ear length are decreased when the maize plant comprising the loss-of-function allele in the endogenous maize gene (e.g., SEQ ID NO: 1, 3, 5, 7, 9, and/or 11 or an allelic variant thereof) is grown under abiotic stress conditions in comparison to internode length, leaf lamina length, leaf sheath length, leaf dry weight, leaf dry matter content, and/or primary ear length for a wild-type control maize plant lacking the loss-of-function allele grown under abiotic stress conditions. Non-limiting examples of abiotic stress include drought, cold, heat, salt, shade, nutrient deficiency, and high planting density. In certain embodiments, the internode length, leaf lamina length, leaf sheath length, leaf dry weight, leaf dry matter content, and/or primary ear length are decreased when the maize plant comprising the loss-of-function allele in the endogenous maize gene (e.g., SEQ ID NO: 1, 3, 5, 7, 9, and/or 11 or an allelic variant thereof) is grown under drought conditions in comparison to internode length, leaf lamina length, leaf sheath length, leaf dry weight, leaf dry matter content, and/or primary ear length for a wild-type control maize plant lacking the loss-of-function allele grown under drought conditions.

In certain embodiments, the stem fresh weight of a maize plant comprising the loss-of-function allele of the endogenous maize gene (e.g., SEQ ID NO: 1, 3, 5, 7, 9, and/or 11 or an allelic variant thereof) is increased in comparison to the stem fresh weight of a wild-type control maize plant lacking the loss-of-function allele. In certain embodiments, the stem fresh weight is increased by at least about 2%, 3%, 4%, 5%, 6%, 7%, 8%, 9%, 10%, 12%, 15%, 18%, 20%, 25%, 30%, 35%, 40%, 45%, or 50% in comparison to the stem fresh weight of the corresponding wild-type control maize plant lacking the loss-of-function allele. In certain embodiments, the stem dry weight of a maize plant comprising the loss-of-function allele of the endogenous maize gene (e.g., SEQ ID NO: 1, 3, 5, 7, 9, and/or 11 or an allelic variant thereof) is increased in comparison to the stem dry weight of a wild-type control maize plant lacking the loss-of-function allele. In certain embodiments, the stem dry weight is increased by at least about 2%, 3%, 4%, 5%, 6%, 7%, 8%, 9%, 10%, 12%, 15%, 18%, 20%, 25%, 30%, 35%, 40%, 45%, or 50% in comparison to the stem dry weight of the corresponding wild-type control maize plant lacking the loss-of-function allele. In certain embodiments, the leaf dry weight of a maize plant comprising the loss-of-function allele of the endogenous maize gene (e.g., SEQ ID NO: 1, 3, 5, 7, 9, and/or 11 or an allelic variant thereof) is increased in comparison to the leaf dry weight of a wild-type control maize plant lacking the loss-of-function allele. In certain embodiments, the leaf dry weight is increased by at least about 2%, 3%, 4%, 5%, 6%, 7%, 8%, 9%, 10%, 12%, 15%, 18%, 20%, 25%, 30%, 35%, 40%, 45%, or 50% in comparison to the leaf dry weight of the corresponding wild-type control maize plant lacking the loss-of-function allele. In certain embodiments, the leaf sheath length of a maize plant comprising the loss-of-function allele of the endogenous maize gene (e.g., SEQ ID NO: 1, 3, 5, 7, 9, and/or 11 or an allelic variant thereof) is increased in comparison to the leaf sheath length of a wild-type control maize plant lacking the loss-of-function allele. In certain embodiments, the leaf sheath length is increased by at least about 2%, 3%, 4%, 5%, 6%, 7%, 8%, 9%, 10%, 12%, 15%, 18%, 20%, 25%, 30%, 35%, 40%, 45%, or 50% in comparison to the leaf sheath length of the corresponding wild-type control maize plant lacking the loss-of-function allele. In certain embodiments, the leaf lamina length of a maize plant comprising the loss-of-function allele of the endogenous maize gene (e.g., SEQ ID NO: 1, 3, 5, 7, 9, and/or 11 or an allelic variant thereof) is increased in comparison to the leaf lamina length of a wild-type control maize plant lacking the loss-of-function allele. In certain embodiments, the leaf lamina length is increased by at least about 2%, 3%, 4%, 5%, 6%, 7%, 8%, 9%, 10%, 12%, 15%1, 18%, 20%, 25%, 30%, 35%, 40%, 45%, or 50% in comparison to the leaf lamina length of the corresponding wild-type control maize plant lacking the loss-of-function allele. In certain embodiments, the primary ear leaf width of a maize plant comprising the loss-of-function allele of the endogenous maize gene (e.g., SEQ ID NO: 1, 3, 5, 7, 9, and/or 11 or an allelic variant thereof) is increased in comparison to the primary ear leaf width of a wild-type control maize plant lacking the loss-of-function allele. In certain embodiments, the primary ear leaf width is increased by at least about 2%, 3%, 4%, 5%, 6%, 7% 8%, 9%10%1, 12%, 15%, 18%, 20%, 25%, 30%, 35%, 40%, 45%, or 50% in comparison to the primary ear leaf width of the corresponding wild-type control maize plant lacking the loss-of-function allele.

In certain embodiments, the stem fresh weight, stem dry weight, leaf dry weight, leaf sheath length, leaf lamina length, and/or primary ear leaf width are increased when the maize plant comprising the loss-of-function allele in the endogenous maize gene (e.g., SEQ ID NO: 1, 3, 5, 7, 9, and/or 11 or an allelic variant thereof) is grown under abiotic stress conditions in comparison to stem fresh weight, stem dry weight, leaf dry weight, leaf sheath length, leaf lamina length, and/or primary ear leaf width for a wild-type control maize plant lacking the loss-of-function allele grown under abiotic stress conditions. Non-limiting examples of abiotic stress include drought, cold, heat, salt, shade, nutrient deficiency, and high planting density. In certain embodiments, the stem fresh weight, stem dry weight, leaf dry weight, leaf sheath length, leaf lamina length, and/or primary ear leaf width are increased when the maize plant comprising the loss-of-function allele in the endogenous maize gene (e.g., SEQ ID NO: 1, 3, 5, 7, 9, and/or 11 or an allelic variant thereof) is grown under drought conditions in comparison to stem fresh weight, stem dry weight, leaf dry weight, leaf sheath length, leaf lamina length, and/or primary ear leaf width for a wild-type control maize plant lacking the loss-of-function allele grown under drought conditions.

Biological samples and commodity plant products obtained from maize plants or maize plant parts comprising a loss-of-function allele of the endogenous maize gene (e.g., SEQ ID NO: 1, 3, 5, 7, 9, and/or 11 or an allelic variant thereof) as well as methods for making such products are provided. In certain embodiments, the commodity products are processed products are made from the maize plant or its seeds, including: (a) maize seed meal (defatted or non-defatted); (b) extracted maize proteins, oils, sugars, syrups, and starches; (c) maize fermentation products; (d) maize based animal feed or human food products (e.g., feed and food comprising maize seed meal (defatted or non-defatted) and other ingredients (e.g., other cereal grains, other seed meal, other protein meal, other oil, other starch, other sugar, a binder, a preservative, a humectant, a vitamin, and/or mineral); (e) a pharmaceutical; (f) raw or processed biomass (e.g., cellulosic and/or lignocellulosic material; silage); and (g) various industrial products.

Also provided herein are methods of treating the maize seeds of the disclosure and the resultant treated seeds. Seeds can be treated with such fertilizers, biological agents, nematicides, insecticides, and fungicides by methods including in-furrow applications or by coating (e.g., with a drum coater, rotary coater, tumbling drum, fluidized bed, and/or spouted bed apparatus).

Methods and compositions including various binders, fillers, film coats, and active ingredients such as fertilizers, surfactants, plant growth regulators, crop desiccants, fungicides, bacteriocides, bacteriostats, insecticides, and insect repellants for coating seeds that can be adapted for use with seeds provided herein are disclosed in U.S. patent Ser. No. 10/745,578, which is incorporated herein by reference in its entirety.

The disclosure also provides a method of making a maize plant comprising a loss-of-function allele of the endogenous maize gene of SEQ ID NO: 1, 3, 5, 7, 9, and/or 11 or an allelic variant thereof. In certain embodiments, the methods can comprise making a deletion, an insertion and/or a substitution which results in a loss-of-function allele of the endogenous maize gene. Gene editing molecules of use in methods provided herein include molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA. In certain embodiments, the loss-of-function allele results from introduction of a DSB at a target site in the endogenous maize gene (e.g., SEQ ID NO: 1, 3, 5, 7, 9, and/or 11 or an allelic variant thereof) to induce non-homologous end joining (NHEJ) at the site of the break followed by recovery of desired loss-of-function alleles. In certain embodiments, the loss-of-function allele results from introduction of a DSB at a target site in the endogenous maize gene (e.g., SEQ ID NO: 1, 3, 5, 7, 9, and/or 11 or an allelic variant thereof) followed by homology-directed repair (HDR), microhomology-mediated end joining (MMEJ), or NHEJ to introduce a desired donor or other DNA template polynucleotide at the DSB, followed by recovery of the desired loss-of-function allele.

Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) optionally donor DNA template polynucleotides suitable for insertion at a break in genomic DNA by homology-directed repair (HDR) or microhomology-mediated end joining (MIMEJ); and (e) optionally other DNA templates (e.g., dsDNA, ssDNA, or combinations thereof) suitable for insertion at a break in genomic DNA (e.g., by non-homologous end joining (NHEJ).

In certain embodiments, the loss-of-function allele of the endogenous maize gene and plant cells, parts including seeds, and plants comprising the loss-of-function allele of the endogenous maize gene are generated by CRISPR technology. CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616.

Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). All of the patent publications referenced in this paragraph are incorporated herein by reference in their entirety. In certain embodiments, an RNA-guided endonuclease that leaves a blunt end following cleavage of the target site is used.

Blunt-end cutting RNA-guided endonucleases include Cas9, Cas12c, Cas12i, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e.

Guide RNA molecules comprising a spacer RNA molecule which targets the endogenous maize gene of SEQ ID NO: 1, 3, 5, 7, 9, 11, or an allelic variant thereof are provided. In certain embodiments, the spacer RNA molecule comprises the RNA encoded by SEQ ID NO: 13, 14, 15, 16, 17, or 18. Guide RNAs comprising a spacer RNA molecule encoded by SEQ ID NO: 13, 14, 15, 16, 17, or 18 can be used in conjunction with a Cas9 nuclease to generate a loss-of-function allele of the endogenous maize gene of SEQ ID NO: 1, 3, 5, 7, 9, and/or 11, or an allelic variant thereof.

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., maize plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, maize plants or maize plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPRI), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif.

Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

In certain embodiments, the loss-of-function allele of the endogenous maize gene (e.g., SEQ ID NO: 1, 3, 5, 7, 9, and/or 11 or an allelic variant thereof) and plant cells, parts including seeds, and plants comprising the loss-of-function allele of the endogenous maize gene (e.g., SEQ ID NO: 1, 3, 5, 7, 9, and/or 11 or an allelic variant thereof) are generated by use of zinc finger nucleases or zinc finger nickases. Zinc-finger nucleases are site-specific endonucleases comprising two protein domains: a DNA-binding domain, comprising a plurality of individual zinc finger repeats that each recognize between 9 and 18 base pairs, and a DNA-cleavage domain that comprises a nuclease domain (typically Fokl). The cleavage domain dimerizes in order to cleave DNA; therefore, a pair of ZFNs are required to target non-palindromic target polynucleotides. In certain embodiments, zinc finger nuclease and zinc finger nickase design methods which have been described (Urnov et al. (2010) Nature Rev. Genet., 11:636-646; Mohanta et al. (2017) Genes vol. 8,12: 399; Ramirez et al. Nucleic Acids Res. (2012); 40(12): 5560-5568; Liu et al. (2013) Nature Communications, 4: 2565) can be adapted for use in the methods set forth herein. The zinc finger binding domains of the zinc finger nuclease or nickase provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) can be adapted for use in the methods described herein. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fokl. This endonuclease must dimerize to cleave DNA. Thus, cleavage by Fokl as part of a ZFN requires two adjacent and independent binding events, which must occur in both the correct orientation and with appropriate spacing to permit dimer formation. The requirement for two DNA binding events enables more specific targeting of long and potentially unique recognition sites. Fokl variants with enhanced activities have been described and can be adapted for use in the methods described herein; see, e.g., Guo et al. (2010) J. Mol. Biol., 400:96-107.

In certain embodiments, the loss-of-function allele of the endogenous maize gene (e.g., SEQ ID NO: 1, 3, 5, 7, 9, and/or 11 or an allelic variant thereof) and plant cells, parts including seeds, and plants comprising the loss-of-function allele of the endogenous maize gene (e.g., SEQ ID NO: 1, 3, 5, 7, 9, and/or 11 or an allelic variant thereof) are generated by use of TAL-effector nucleases or TALENs. Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fokl, can be conveniently used. Methods for use of TALENs in plants have been described and can be adapted for use in the methods described herein, see Mahfouz et al. (2011) Proc. Natl. Acad. Sci. USA, 108:2623-2628; Mahfouz (2011) GM Crops, 2:99-103; and Mohanta et al. (2017) Genes vol. 8,12: 399). TALE nickases have also been described and can be adapted for use in methods described herein (Wu et al.; Biochem Biophys Res Commun. (2014); 446(1):261-6; Luo et al; Scientific Reports 6, Article number: 20657 (2016)).

Various treatments can be used for delivery of gene editing molecules and/or other molecules to a plant cell. In certain embodiments, one or more treatments is employed to deliver the gene editing or other molecules (e.g., comprising a polynucleotide, polypeptide or combination thereof) into a plant cell, e.g., through barriers such as a cell wall, a plasma membrane, a nuclear envelope, and/or other lipid bilayer. In certain embodiments, a polynucleotide-, polypeptide-, or RNP (ribonucleoprotein)-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing molecule-containing composition. In certain embodiments, the composition is delivered using negative or positive pressure, for example, using vacuum infiltration or application of hydrodynamic or fluid pressure. In certain embodiments, the composition is introduced into a plant cell or plant protoplast, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the composition to a eukaryotic cell, plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In certain embodiments, the composition is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the genome editing molecules (e.g., RNA dependent DNA endonuclease, RNA dependent DNA binding protein, RNA dependent nickase, ABE, or CBE, and/or guide RNA); see, e.g., Broothaerts et al. (2005) Nature, 433:629-633).

Any of these techniques or a combination thereof are alternatively employed on a plant explant, plant part or tissue or intact plant (or seed) from which a plant cell is optionally subsequently obtained or isolated; in certain embodiments, the composition is delivered in a separate step after the plant cell has been isolated.

In certain embodiments, the methods for generating the maize plant cell, maize plant parts, or maize plants comprise: (i) screening a population of maize plant cells, parts, or plants for the presence of a loss-of-function allele of the endogenous maize gene of SEQ ID NO: 1, 3, 5, 7, 9, and/or 11 or an allelic variant thereof; and (ii) isolating a maize plant cell, maize plant part, or maize plant comprising a loss-of-function allele of the endogenous maize gene of SEQ ID NO: 1, 3, 5, 7, 9, and/or 11 or an allelic variant thereof.

In certain embodiments, the population of maize plant cells, parts, or plants which are screened for the presence of a loss-of-function allele in the endogenous maize gene (e.g., SEQ ID NO: 1, 3, 5, 7, 9, and/or 11 or an allelic variant thereof) are first pre-screened by screening of phenotypic characteristics plants having a loss-of-function allele in the endogenous maize gene.

In certain embodiments, such phenotypic characteristics include increased water use efficiency and/or drought tolerance in comparison to water use efficiency and/or drought tolerance for a wild-type control maize plant lacking the loss-of-function allele. In certain embodiments, such phenotypic characteristics include decreased internode length, leaf lamina length, leaf sheath length, leaf dry weight, leaf dry matter content, and/or primary ear length in comparison to internode length, leaf lamina length, leaf sheath length, leaf dry weight, leaf dry matter content, and/or primary ear length for a wild-type control maize plant lacking the loss-of-function allele.

In certain embodiments, such phenotypic characteristics include stem fresh weight, stem dry weight, leaf dry weight, leaf sheath length, leaf lamina length, and/or primary ear leaf width in comparison to stem fresh weight, stem dry weight, leaf dry weight, leaf sheath length, leaf lamina length, and/or primary ear leaf width for a wild-type control maize plant lacking the loss-of-function allele. In certain embodiments, the screened and control plants are grown under abiotic stress conditions (e.g., drought, cold, heat, or salt stress). In certain embodiments, plants exhibiting one or more of the aforementioned phenotypic characteristics are then subjected to screening for the presence of a loss-of-function allele in the endogenous maize gene, and maize plants comprising a loss-of-function allele in the endogenous maize gene are identified and/or selected.

In certain embodiments, the population of maize plant cells, parts, or plants which are screened for the presence of a loss-of-function allele in the endogenous maize gene (e.g., SEQ ID NO: 1, 3, 5, 7, 9, and/or 11 or an allelic variant thereof) have been subjected to one or more mutagenesis treatments. Loss-of-function alleles of the endogenous maize gene can be generated by mutagenesis methods known in the art, such as chemical mutagenesis or radiation mutagenesis. Suitable chemical mutagens include ethyl methanesulfonate (EMS), sodium azide, methylnitrosourea (MNU), and diepoxybutane (DEB). Suitable radiation includes x-rays, fast neutron radiation, and gamma radiation.

Maize plant cells, parts, or plants comprising a loss-of-function allele of the endogenous maize gene (e.g., SEQ ID NO: 1, 3, 5, 7, 9, and/or 11 or an allelic variant thereof) can be generated using mutagenesis and identified by TILLING (Targeting Induced Local Lesions IN Genomes) or identified using EcoTILLING. TILLING is a general reverse genetics technique that uses mutagenesis methods to create libraries of mutagenized individuals that are later subjected to high throughput screens for the discovery of mutations. In addition to allowing efficient detection of induced mutations, high-throughput TILLING technology is ideal for the detection of natural mutations. EcoTILLING is a method that uses TILLING techniques to look for natural mutations in individuals (Barkley and Wang. Current genomics vol. 9,4 (2008): 212-26. doi:10.2174/138920208784533656). Identified mutations can then be introduced into desirable genetic backgrounds by crossing the mutant with a plant of the desired genetic background and performing a suitable number of backcrosses to cross out the originally undesired parent background. A more detailed description of methods and compositions for TILLING are disclosed in US Patent Application Publication 2004/0053236 A1, which is incorporated herein by reference in its entirety and can be adapted for use in the methods provided herein for identifying maize plant cells, parts, or plants comprising a loss-of-function allele of the endogenous maize gene.

In certain embodiments, the screening comprises analyzing water use efficiency and/or drought tolerance of one or more candidate plants or one or more candidate plant populations. In certain embodiments, the screening comprises analyzing internode length, stem fresh weight, stem dry weight, leaf lamina length, leaf sheath length, leaf dry weight, leaf dry matter content, primary ear leaf width, and/or primary ear length in one or more candidate plants or one or more candidate plant populations. In these embodiments, a decrease in internode length, leaf lamina length, leaf sheath length, leaf dry weight, leaf dry matter content, and/or primary ear length; or an increase in stem fresh weight, stem dry weight, leaf dry weight, leaf sheath length, leaf lamina length, and/or primary ear leaf width in comparison to a wild-type control maize plant lacking the loss-of-function allele is indicative of a maize plant cell, plant part, or plant comprising the loss-of-function allele. In certain embodiments, the screening is conducted on a population of plants grown under abiotic stress conditions. Suitable examples of abiotic stress conditions include drought, salt, cold, heat, salt, shade, nutrient deficiency, and high planting density.

This disclosure is also directed to methods for producing a maize plant having a loss-of-function allele of the endogenous maize gene (e.g., SEQ ID NO: 1, 3, 5, 7, 9, and/or 11 or an allelic variant thereof) by crossing a first parent maize plant with a second parent maize plant wherein the first or second parent maize plant comprises the loss-of-function allele. Further, both the first and second parent maize plants can comprise the loss-of-function allele. Any such methods using a maize plant comprising the loss-of-function allele are part of this disclosure: selfing, backcrosses, hybrid production, crosses to populations, and the like. All plants produced using a maize plant comprising the loss-of-function allele as a parent are within the scope of this disclosure, including plants derived from a maize plant having the loss-of-function allele. Also provided are the F₁ progeny maize plants produced from the crossing of a maize plant comprising the loss-of-function allele with any other maize plant, F₁ seed, and various parts of the F₁ maize plant. The following describes breeding methods that can be used with maize plants of the disclosure in the development of further maize plants. One such embodiment is a method for developing a progeny maize plant in a maize plant breeding program comprising: obtaining the maize plant, or its parts, comprising a loss-of-function allele of the endogenous maize gene and utilizing the plant or plant parts as a source of breeding material; and selecting a progeny plant having the loss-of-function allele. Breeding steps that can be used in the maize plant breeding program include pedigree breeding, backcrossing, mutation breeding, and recurrent selection. In conjunction with these steps, techniques such as restriction fragment polymorphism enhanced selection, marker-assisted selection (for example SNP or SSR markers), and the making of double haploids can be utilized.

Field crops are bred through techniques that take advantage of the plant's method of pollination. A maize plant of the disclosure can be self-pollinated, sib-pollinated, or cross pollinated to create a pedigree maize plant. A plant is self-pollinated if pollen from one flower is transferred to the same or another flower of the same plant. A plant is sib-pollinated when individuals within the same family or variety are used for pollination. A plant is cross-pollinated if the pollen comes from a flower on a different plant from a different family or variety. The terms “cross-pollination” and “out-cross” as used herein do not include self-pollination or sib-pollination. Maize can be bred by both self-pollination and cross-pollination techniques. Maize has separate male and female flowers on the same plant, located on the tassel and the ear, respectively. Natural pollination occurs in maize when wind blows pollen from the tassels to the silks that protrude from the tops of the ears.

Any other suitable breeding, selection, or growing methods may be used. Choice of the particular breeding or selection method will vary depending on environmental factors, population size, and the like.

Inbred and hybrid maize plants and seeds comprising a loss-of-function allele of the endogenous maize gene (e.g., SEQ ID NO: 1, 3, 5, 7, 9, and/or 11 or an allelic variant thereof) are provided herein along with methods for making and using such hybrid and inbred seed.

Methods for inbred seed production include selfing inbred maize plants and restricting cross-pollination by any maize plants other than the inbred maize plant. Methods for production of such hybrid seed can comprise crossing elite crop maize plant lines where at least one of the pollen donor or recipient comprises the loss-of-function allele. In certain embodiments, methods of making hybrid seed can comprise crossing elite crop maize plant lines where the pollen recipient comprises the loss-of-function allele and where the pollen recipient is homozygous for the loss-of-function allele. In certain embodiments, methods of making hybrid seed can comprise crossing elite crop maize plant lines where both the pollen donor and recipient comprise the loss-of-function allele and where both the pollen donor and pollen recipient are homozygous for the loss-of-function allele. Methods for hybrid seed production have been disclosed (MacRobert, J. F., P. S. Setimela, J. Gethi, and M. Worku. 2014. Maize Hybrid Seed Production Manual. Mexico, D. F.: CIMMYT) and can be adapted to the production of hybrids disclosed herein.

In certain embodiments, the inbred maize plant, the hybrid maize plant, the pollen donor and/or the pollen recipient can further comprise a transgenic locus which confers a trait (e.g., herbicide tolerance or insect resistance such as coleopteran or lepidopteran insects). Transgenes that can be introduced into the maize plants comprising a loss-of-function allele of the endogenous maize gene (e.g., SEQ ID NO: 1, 3, 5, 7, 9, and/or 11 or an allelic variant thereof) by breeding or by direct transformation include: (i) transgenes that confer insect resistance (e.g., transgenes that produce Bacillus thuringiensis proteins including Cry1Ab, Cry1Ac, Cry1F, Cry2Ab, Cry2Ae, Cry3A, Cry3Bb, Cry9c, Cry34, Cry35, VIP3A, and variants thereof; transgenes that induce insect-inhibitory RNAi responses); and (ii) transgenes that confer tolerance to distinct herbicides (e.g., CP4-EPSPS or other EPSPS genes which confer glyphosate tolerance; PAT or BAR genes which confer resistance to glufosinate herbicides; aad-1 genes which confer resistance to 2,4-D and aryloxyphenoxypropionate herbicides; DMO genes which confer resistance to dicamba herbicide). Examples of selected transgenic maize plant events which contain transgenes that confer traits such as herbicide tolerance and/or pest tolerance are disclosed in U.S. Pat. Nos. 6,342,660, 7,956,246, 8,575,434, 7,314,970, 8,759,618, 6,852,915, 10316330, 8618358, 8450561, 8686230, 9428765, 8455720, 7897748, 8273959, 8093453, 8502047, and 8466346, which are each incorporated herein by reference in their entireties.

In certain embodiments, maize plants provided herein which a loss-of-function allele of the endogenous maize gene (e.g., SEQ ID NO: 1, 3, 5, 7, 9, and/or 11 or an allelic variant thereof) can further comprise one or more targeted genetic changes introduced by one or more of gene editing molecules or systems. Such targeted genetic changes include those conferring traits such as improved yield, improved food and/or feed characteristics (e.g., improved oil, starch, protein, or amino acid quality or quantity), improved nitrogen use efficiency, improved biofuel use characteristics, herbicide tolerance (e.g., by targeting endogenous ALS, EPSPS, HPPD, or other herbicide target genes), delayed flowering, non-flowering, increased biotic stress resistance (e.g., resistance to insect, nematode, bacterial, or fungal damage), enhanced lodging resistance, enhanced growth rate, enhanced biomass, enhanced branching, delayed flowering time, delayed senescence, increased flower number, improved architecture for high density planting, improved photosynthesis, increased root mass, increased cell number, improved seedling vigor, improved seedling size, increased rate of cell division, improved metabolic efficiency, and increased meristem size in comparison to a control maize plant lacking the targeted genetic change. Types of targeted genetic changes that can be introduced include insertions, deletions, and substitutions of one or more nucleotides in the maize plant genome.

Sites in endogenous maize plant genes for the targeted genetic changes include promoter, coding, and non-coding regions (e.g., 5′ UTRs, introns, splice donor and acceptor sites and 3′ UTRs). Non-limiting examples of target maize genes that can be subjected to targeted gene edits to confer useful traits include quality and herbicide tolerance traits. In certain embodiments, such targeted genetic changes can be combined with plants which comprise a loss-of-function allele of the endogenous maize gene of SEQ ID NO: 1, 3, 5, 7, 9, and/or 11 or an allelic variant thereof. Such breeding techniques include crossing and/or introgression by backcrossing to a recurrent parent. In such crosses, the plants which comprise the loss-of-function allele of the endogenous maize gene (e.g., SEQ ID NO: 1, 3, 5, 7, 9, and/or 11 or an allelic variant thereof) can be either a pollen donor or recipient. In certain embodiments, plants which comprise the loss-of-function allele can be used as the recurrent parent in such backcrosses to introgress the targeted genetic change into plant germplasm comprising the loss-of-function allele. In certain embodiments, plants which comprise the target genetic change(s) can be used as the recurrent parent in such backcrosses to introgress the genomic region comprising the loss-of-function allele into plant germplasm comprising the target genetic change(s).

Methods for determining whether a maize plant cell, plant part, or plant comprises a loss-of-function allele of the endogenous maize gene (e.g., SEQ ID NO: 1, 3, 5, 7, 9, and/or 11 or an allelic variant thereof) are provided. Methods for determining the presence or absence of the loss-of-function allele can be used in, for example, breeding programs for identification, selection, introgression, and the like.

In certain embodiments, the methods comprise analyzing a polynucleotide comprising a portion of SEQ ID NO: 1, 3, 5, 7, 9, and/or 11 or an allelic variant thereof or analyzing an RNA encoded by a portion of SEQ ID NO: 1, 3, 5, 7, 9, and/or 11 or an allelic variant thereof from the plant cell, plant part, or plant. In certain embodiments, an insertion, deletion, and/or substitution of one or more nucleotides in the polynucleotide or RNA is indicative of the presence of the loss-of-function allele. Detection of the loss-of-function allele in a nucleic acid sample (e.g., DNA, RNA, or cDNA) can be achieved by any combination of nucleic acid amplification (e.g., PCR amplification), hybridization, sequencing, and/or mass-spectrometry based techniques. In certain embodiments, such detection is achieved by amplification and/or hybridization-based detection methods using a primer (e.g., selective amplification primers) and/or probe (e.g., capable of selective hybridization or generation of a specific primer extension product) which specifically recognizes the endogenous maize gene (e.g., a portion of SEQ ID NO: 1, 3, 5, 7, 9, and/or 11 or an allelic variant thereof). Such primers and/or probes can comprise or consist of about 15, 20, 25, 30, 40, 45 or 50 more contiguous nucleotides of SEQ ID NO: 1, 3, 5, 7, 9, and/or 11 or an allelic variant thereof. In certain embodiments, the primers or probes can comprise or consist of about 10 to 50 contiguous nucleotides, about 10 to 40 contiguous nucleotides, about 10 to 30 contiguous nucleotides or about 15 to 30 contiguous nucleotides of SEQ ID NO: 1, 3, 5, 7, 9, and/or 11 or an allelic variant thereof. In certain embodiments, the hybridization probes (e.g., polynucleotides comprising at least about 15 to 30 base pairs of SEQ ID NO: 1, 3, 5, 7, 9, and/or 11 or an allelic variant thereof) can comprise detectable labels (e.g., fluorescent, radioactive, epitope, and chemiluminescent labels). In certain embodiments, the endogenous maize gene can be directly sequenced using nucleic acid sequencing technologies, including whole genome sequencing.

In certain embodiments, the methods comprise analyzing a polypeptide encoded by SEQ ID NO: 1, 3, 5, 7, 9, and/or 11, a portion thereof, or an allelic variant thereof from the maize plant cell, plant part, or plant. In certain embodiments, an insertion, deletion, and/or substitution of one or more amino acid residues of the polypeptide or a change in the biologic or biochemical activity of the polypeptide is indicative of the presence of the loss-of-function allele. Detection of the loss-of-function allele based on the polypeptide can be determined by methods well known in the art such as activity assays, western blots using antibodies capable of specifically binding the polypeptide, enzyme-linked immunosorbent assays (ELISA), radioimmunoassays (RIA), immunohistochemistry, immunocytochemistry, immunofluorescence, and the like.

In certain optional embodiments, the maize plant cells disclosed herein are non-regenerable maize plant cells. In certain optional embodiments provided herein, the maize plant cells, plant propagules (e.g., a seed, seedling, ovule, embryo, pollen, root, stem, leaf, shoot, explant, or callus), and plants provided herein are not produced by an exclusively biological process. In certain optional embodiments provided herein, the methods for producing maize plant cells, plant propagules (e.g., a seed, seedling, ovule, embryo, pollen, root, stem, leaf, shoot, explant, or callus), and plants provided herein are not exclusively biological processes.

The following numbered embodiments also form part of the present disclosure:

1. A maize plant comprising a loss-of-function allele of the endogenous maize gene of SEQ ID NO: 1, 3, 5, 7, 9, and/or 11, or an allelic variant thereof.

2. The maize plant of embodiment 1, wherein the loss-of-function allele comprises an amorphic allele of the endogenous maize gene.

3. The maize plant of embodiment 1 or embodiment 2, wherein the loss-of-function allele comprises a hypomorphic allele of the endogenous maize gene.

4. The maize plant of any one of embodiments 1-3, wherein the plant is homozygous for the loss-of-function allele of the endogenous maize gene.

5. The maize plant of any one of embodiments 1-4, wherein the loss-of-function allele comprises a frameshift mutation, a missense mutation, or a nonsense mutation in the endogenous maize gene.

6. The maize plant of any one of embodiments 1-5, wherein the plant comprises a loss-of-function allele of the endogenous maize gene of SEQ ID NO: 1, or an allelic variant thereof.

7. The maize plant of any one of embodiments 1-6, wherein the plant comprises a loss-of-function allele of the endogenous maize gene of SEQ ID NO: 3, or an allelic variant thereof.

8. The maize plant of any one of embodiments 1-7, wherein the plant comprises a loss-of-function allele of the endogenous maize gene of SEQ ID NO: 5, or an allelic variant thereof.

9. The maize plant of any one of embodiments 1-8, wherein the plant comprises a loss-of-function allele of the endogenous maize gene of SEQ ID NO: 7, or an allelic variant thereof.

10. The maize plant of any one of embodiments 1-9, wherein the plant comprises a loss-of-function allele of the endogenous maize gene of SEQ ID NO: 9, or an allelic variant thereof.

11. The maize plant of any one of embodiments 1-10, wherein the plant comprises a loss-of-function allele of the endogenous maize gene of SEQ ID NO: 11, or an allelic variant thereof.

12. The maize plant of any one of embodiments 1-11, wherein water use efficiency of the maize plant is increased in comparison to water use efficiency of a control maize plant lacking the loss-of-function allele.

13. The maize plant of any one of embodiments 1-12, wherein drought tolerance of the maize plant is increased in comparison to drought tolerance of a control maize plant lacking the loss-of-function allele.

14. The maize plant of any one of embodiments 1-12, wherein internode length, leaf lamina length, leaf sheath length, leaf dry weight, leaf dry matter content, and/or primary ear length are decreased in comparison to internode length, leaf lamina length, leaf sheath length, leaf dry weight, leaf dry matter content, and/or primary ear length for a control maize plant lacking the loss-of-function allele.

15. The maize plant of any one of embodiments 1-14, wherein internode length, leaf lamina length, leaf sheath length, leaf dry weight, leaf dry matter content, and/or primary ear length are decreased under drought conditions.

16. The maize plant of any one of embodiments 1-15, wherein stem fresh weight, stem dry weight, leaf dry weight, leaf sheath length, leaf lamina length, and/or primary ear leaf width are increased in comparison to stem fresh weight, stem dry weight, leaf dry weight, leaf sheath length, leaf lamina length, and/or primary ear leaf width for a control maize plant lacking the loss-of-function allele.

17. The maize plant of any one of embodiments 1-16, wherein stem fresh weight, stem dry weight, leaf dry weight, leaf sheath length, leaf lamina length, and/or primary ear leaf width are increased under drought conditions.

18. The maize plant of any one of embodiments 1-17, with the proviso that the plant is not exclusively produced by an essentially biological method.

19. The maize plant of any one of embodiments 1-18, wherein the plant comprises elite germplasm.

20. The maize plant of any one of embodiments 1-19, wherein the plant further comprises: (i) one or more mutations in a distinct maize gene; and/or (ii) one or more transgenes, optionally wherein the transgene encodes a protein or RNA conferring herbicide tolerance or pest tolerance.

21. A maize plant part of the plant of any one of embodiments 1-20, wherein the maize plant part comprises the loss-of-function allele.

22. The maize plant part of embodiment 21, wherein the part is a seed, stalk, stem, pollen, or leaf.

23. The maize plant part of embodiment 21 or embodiment 22, wherein the part is a seed.

24. The maize plant part of any one of embodiments 21-23, wherein the seed further comprises at least a partial coating of a composition comprising a biological agent, nematicide, insecticide, or fungicide.

25. A maize plant cell of the plant of any one of embodiments 1-20.

26. A method of producing maize seed, comprising growing the maize plant of any one of embodiments 1-20 and harvesting seed therefrom.

27. A biological sample comprising a nucleic acid containing a loss-of-function allele of the endogenous maize gene of SEQ ID NO: 1, 3, 5, 7, 9, and/or 11, or an allelic variant thereof.

28. The biological sample of embodiment 27, wherein the biological sample comprises material obtained from the maize plant of any one of embodiments 1-20 or a part thereof, optionally wherein the part is a seed.

29. The biological sample of embodiment 27 or embodiment 28, wherein the biological sample is non-regenerable.

30. The biological sample of any one of embodiments 27-29, wherein the biological sample comprises maize seed meal.

31. A method of producing a commodity maize plant product, the method comprising: (i) processing the maize plant of any one of embodiments 1-20 or a maize seed obtained therefrom; and (ii) recovering the commodity maize plant product from the processed maize plant or maize seed.

32. The method of embodiment 31, wherein the commodity maize plant product is seed meal, starch, syrup, silage, oil, or protein.

33. The method of embodiment 31 or embodiment 32, wherein the commodity maize plant product comprises a detectable amount of a DNA molecule comprising the loss-of-function allele of the endogenous maize gene.

34. A guide RNA molecule comprising a spacer RNA molecule that targets the endogenous maize gene of SEQ ID NO: 1, 3, 5, 7, 9, 11, or an allelic variant thereof.

35. The guide RNA molecule of embodiment 34, wherein the spacer RNA molecule comprises the RNA encoded by SEQ ID NO: 13, 14, 15, 16, 17, or 18.

36. A method for generating the maize plant of any one of embodiments 1-20 comprising introducing a loss-of-function allele in the endogenous maize gene of SEQ ID NO: 1, 3, 5, 7, 9, and/or 11, or an allelic variant thereof.

37. The method of embodiment 36, wherein the loss-of-function allele is introduced by: (i) directing both: (a) a guide RNA (gRNA) molecule comprising a spacer RNA molecule that targets the endogenous maize gene of SEQ ID NO: 1, 3, 5, 7, 9, and/or 11, or an allelic variant thereof, and (b) an RNA dependent endonuclease (RDE) that recognizes the gRNA molecule to the genome of a target maize plant cell; and (ii) isolating a maize plant comprising the loss-of-function allele from the maize plant cell.

38. The method of embodiment 36 or embodiment 37, wherein the directing of the gRNA and the RDE to the genome of the target maize plant cell comprises introducing the gRNA, the RDE, a gRNA/RDE complex, a nucleic acid encoding the gRNA, and/or a nucleic acid encoding the RDE into the target maize plant cell.

39. The method of any one of embodiments 36-38, wherein the spacer RNA molecule comprises the RNA encoded by SEQ ID NO: 13, 14, 15, 16, 17, or 18.

40. The method of any one of embodiments 36-39, wherein water use efficiency of the maize plant is increased in comparison to water use efficiency of a control maize plant lacking the loss-of-function allele.

41. The method of any one of embodiments 36-40, wherein drought tolerance of the maize plant is increased in comparison to drought tolerance of a control maize plant lacking the loss-of-function allele.

42. The method of any one of embodiments 36-41, wherein internode length, leaf lamina length, leaf sheath length, leaf dry weight, leaf dry matter content, and/or primary ear length are decreased in comparison to internode length, leaf lamina length, leaf sheath length, leaf dry weight, leaf dry matter content, and/or primary ear length for a control maize plant lacking the loss-of-function allele.

43. The method of any one of embodiments 36-42, wherein internode length, leaf lamina length, leaf sheath length, leaf dry weight, leaf dry matter content, and/or primary ear length are decreased under drought conditions.

44. The method of any one of embodiments 36-43, wherein stem fresh weight, stem dry weight, leaf dry weight, leaf sheath length, leaf lamina length, and/or primary ear leaf width are increased in comparison to stem fresh weight, stem dry weight, leaf dry weight, leaf sheath length, leaf lamina length, and/or primary ear leaf width for a control maize plant lacking the loss-of-function allele.

45. The method of any one of embodiments 36-44, wherein stem fresh weight, stem dry weight, leaf dry weight, leaf sheath length, leaf lamina length, and/or primary ear leaf width are increased under drought conditions.

46. The method of any one of embodiments 36-45, wherein the loss-of-function allele is introduced by crossing a maize plant comprising the loss-of-function allele with a second maize plant and harvesting F1 seed comprising the loss-of-function allele, thereby producing progeny maize seed comprising the loss-of-function allele.

47. A method for determining whether a maize plant cell, plant part, plant, or biological sample obtained therefrom comprises a loss-of-function allele of the endogenous maize gene of SEQ ID NO: 1, 3, 5, 7, 9, and/or 11, or an allelic variant thereof, the method comprising: (i) analyzing a polynucleotide comprising a portion of SEQ ID NO: 1, 3, 5, 7, 9, and/or 11, or an allelic variant thereof, or analyzing an RNA encoded by a portion of SEQ ID NO: 1, 3, 5, 7, 9, and/or 11, or an allelic variant thereof, from the maize plant cell, plant part, plant, or biological sample, wherein an insertion, deletion, and/or substitution of one or more nucleotides in the polynucleotide or RNA is indicative of the presence of the loss-of-function allele in the maize plant cell, plant part, plant, or biological sample; and/or (ii) analyzing a polypeptide encoded by SEQ ID NO: 1, 3, 5, 7, 9, and/or 11, a portion thereof, or an allelic variant thereof from the maize plant cell, plant part, or plant, wherein an insertion, deletion, and/or substitution of one or more amino acid residues of the polypeptide or a change in the biologic or biochemical activity of the polypeptide is indicative of the presence of the loss-of-function allele in the maize plant cell, plant part, plant, or biological sample.

### EXAMPLES

### Example 1: Distinct Growth Recovery Mechanisms Occur after Rehydration Drought-Stressed Plants, Depending on the Growth Phase

Because of the linear organization of the growth-promoting processes of cell division and cell expansion at its basis, the maize leaf is an excellent model system to study the effect of drought on growth. Final leaf length is determined by the maximal growth rate (leaf elongation rate; LER) and the duration of the growth period (leaf elongation duration; LED). Furthermore, phenotyping of two independent recombinant inbred line (RTL) populations has demonstrated that under well-watered conditions, the leaf elongation rate and leaf elongation duration behave as two independent processes, each contributing to the final leaf size.

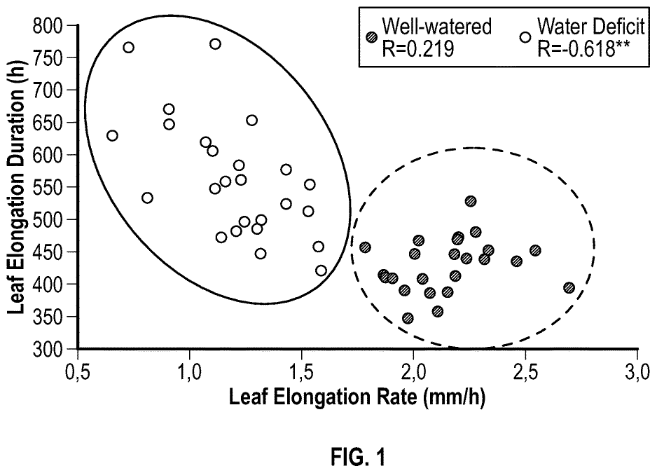

A custom-made, automated platform was used to monitor and water the plants daily to reach a relative gravimetric soil water content of 2.4 (well-watered: WW) or 1.0 (water deficit: WD) g H₂O/g dry soil. The average final length of the fourth leaf of B73 water deficit plants was significantly reduced (21.6%; p=2×10⁻⁹) compared with that of B73 well-watered plants, but no leaf rolling, wilting or senescence was observed. The significant reduction in leaf elongation rate in the early growth phase of the leaf (48% reduction; p=1.1×10⁻⁹) was partly compensated by a 41% prolonged leaf elongation duration (p=0.00025) compared with B73 well-watered plants. The significant anti-correlation (R²=−0.618; p=0.001) between leaf elongation rate and leaf elongation duration in 23 RILs (FIG. 1) from a F12 RIL population derived from a cross between the inbred parents B73 and H99 showed that this compensation mechanism was under genetic control.

Next, whether the prolonged but reduced growth of plants exposed to moderate drought would allow for growth recovery when water became available again was tested. To this end, a rehydration experiment was performed using RIL89, that showed a strongly reduced leaf elongation rate and remarkably extended leaf elongation duration at water deficit conditions.

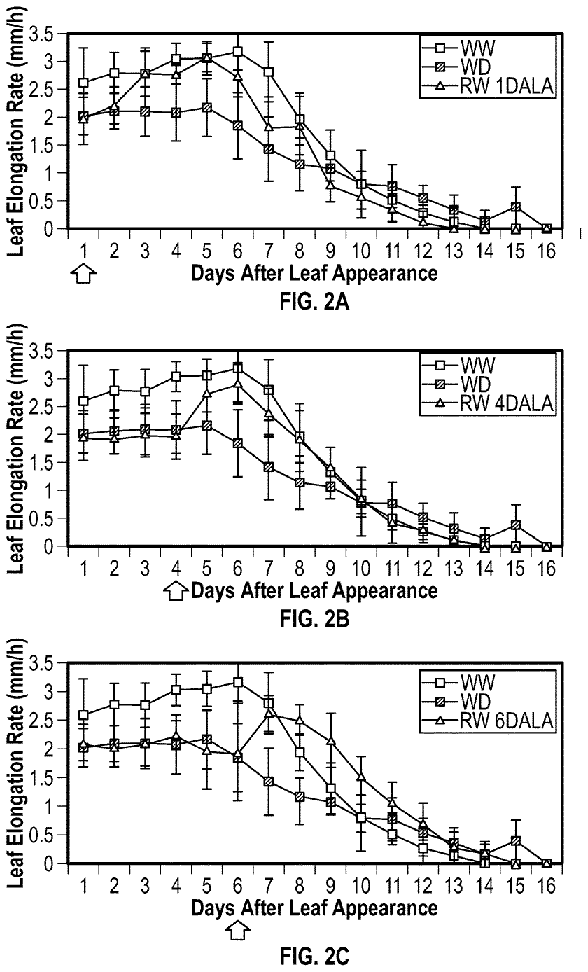

Rehydration up to 4 days after leaf appearance (DALA) or at the end of steady-state growth (6 and 8 days after leaf appearance) had a positive effect on various traits of leaf four, such as final leaf length (FLL), leaf weight, leaf area and leaf width compared with plants continuously exposed to water deficit conditions. However, depending on the timing of rehydration distinct growth recovery mechanisms are operating. Rehydration during steady-state growth largely recovered the leaf elongation rate (FIG. 2A-B) and reduced the leaf elongation duration again to levels observed under well-watered conditions. However, only partial recovery of the leaf elongation rate to well-watered conditions and, in addition, a prolonged leaf elongation duration similar to water deficit conditions was seen when plants were rehydrated at the end of steady-state growth (FIG. 2C). Rehydration after steady-state growth (12 days after leaf appearance), when the leaf elongation rate was already decreasing in well-watered conditions, had no effect on leaf size traits or on the leaf elongation rate and the leaf elongation duration compared with water deficit conditions.

Next, whether the observed developmental time-dependent effects can be generalized to other genotypes and plant growth conditions was investigated. For this, B104 plants were grown in a greenhouse equipped with an automated weighing and watering system for larger plants. Water deficit conditions from V5 onwards, when leaf twelve is already visible in the shoot apical meristem, resulted in a significantly reduced leaf elongation rate (23%, p<0.001) and a significantly increased leaf elongation duration (16%, p<0.001) of leaf 12 compared with well-watered conditions. After leaf 12 appearance, water deficit plants were rehydrated at different time points during the eleven-day steady-state growth period. A complete leaf elongation rate recovery and no significant difference in the leaf elongation duration were observed after rehydration when the twelfth leaf appeared from the pseudo-stem and at two days after leaf appearance compared with well-watered conditions. A partial leaf elongation rate recovery and significantly prolonged leaf elongation duration were observed at later rehydration time points during steady-state growth (at 6 and 9 days after leaf appearance).

In conclusion, these data indicate that there are different growth response mechanisms after rehydration, depending on the growth phase of the leaf. While rehydration around leaf appearance resulted in a restoration of the maximal growth rate, rehydration towards the end of steady-state growth only partially restored leaf elongation rate and prolonged leaf elongation duration. These two different growth recovery mechanisms are conserved across different genotypes, leaves and growing conditions.

### Example 2: The Capacity to Fully Restore Growth after Rehydration Depends on Cell Division Activity

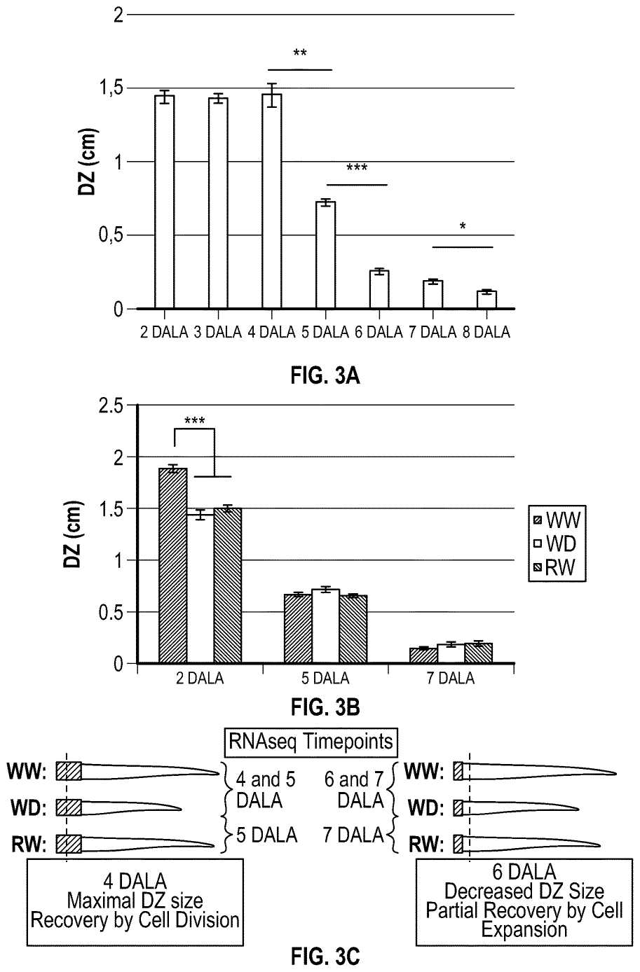

To investigate if the recovery mechanism was dependent on the proliferative phase of the leaf, the size of the division zone (DZ) of RIL89 plants was followed over time under water deficit conditions. The tilting point between the distinct recovery mechanisms (6 days after leaf appearance; FIG. 2B-C) coincided with the moment when the division zone reduces in size (from 5 days after leaf appearance onwards, FIG. 3A). However, rehydration was unable to restore the size of the division zone reduction caused by water deficit at 2 days after leaf appearance (FIG. 3B; Table 1). Therefore, a kinematic analysis was performed to study the contributions of cell division and cell expansion to the distinct recovery mechanisms. The increase in leaf elongation rate after rehydration at 1 and 4 days after leaf appearance was the exclusive result of a significantly increased cell production (17% (p=0.04) and 26% (p=0.01), respectively), whereas cell expansion was not significantly affected (Table 1). Conversely, when water deficit plants were rehydrated at 6 days after leaf appearance, the 11% increase in final length of the fourth leaf (FLL4) originating from the partial increase in leaf elongation rate and prolongation of leaf elongation duration, was caused by a significant increase in mature cell size (28%, p=0.05), whereas cell production was not significantly affected (Table 1).

Together, the data indicated that upon rehydration, two distinct mechanisms of recovery can be triggered: when the division zone is still active, growth recovery was achieved through cell division, but when the division zone is consumed, partial recovery is achieved through cell expansion (FIG. 3C). These data suggest that the ability to maintain the cell division activity for a longer time would be a beneficial trait in water deficit recovery. To test this hypothesis, pGA2ox::PLA1 lines, in which the division zone activity and leaf elongation duration are prolonged were used. Rehydration of water deficit pGA2ox::PLA1 transgenic plants resulted in an almost complete growth recovery (final leaf length) to well-watered conditions up to 6 days after leaf appearance, whereas the growth recovery was only achieved when rehydration occurred at 4 days after leaf appearance in the non-transgenic plants. These data show that division zone activity of water deficit maize leaves is crucial to resume growth after rehydration and that expanding the time window of cell division, by ectopic overexpression of PLA1, increased the plant's opportunity to fully recover from water deficit conditions through their maximal growth rate.

Table 1 shows the effect of water deficit (WD) and rehydration (RW) treatments on cellular growth parameters in RIL89. Differences (in %) between rehydration and water deficit conditions at two, five and seven days after leaf appearance (DALA) were determined for different cellular parameters (n=4, except for WD at 7 DALA n=3; n=7-25 for LER). DZ: division zone, LER: average leaf elongation rate during the steady state growth phase. Statistical significance was determined by a Student's t-test; significant values are indicated in bold. All measurements were performed on the fourth leaf.

TABLE 1

| | % change RW | | % change RW | | % change RW | |
|---|---|---|---|---|---|---|
| | versus WD 2 | | versus WD 5 | | versus WD 7 | |
| Parameters | DALA | P-value | DALA | P-value | DALA | P-value |
| DZ size (mm) | 4.07 | 0.36 | −8.71 | 0.08 | 10.08 | 0.51 |
| Size mature cells (μm) | 3.78 | 0.47 | −2.32 | 0.62 | 27.52 | 0.05 |
| Cell production (cells/h) | 17.01 | 0.04 | 25.98 | 0.01 | 17.62 | 0.17 |
| LER (mm/h) | 11.07 | 0.01 | 22.58 | 0.03 | 51.67 | 6.02E−04 |
| Final leaf size (mm) | 16.64 | 0.02 | 11.73 | 0.05 | 11.25 | 0.13 |

### Example 3: The Two Different Mechanisms of Growth Recovery are Associated with Distinct Transcriptional Reprograming

To study the molecular changes associated with the two different drought-recovery mechanisms an RNA-sequencing (RNA-seq) experiment was performed on the division zone (the basal 5 mm) of the fourth leaf of RIL89 plants at different time points. Well-watered and water deficit samples were harvested four, five, six and seven days after leaf appearance. Plants rehydrated after 4 days were harvested at 5 days after leaf appearance, while plants rehydrated at 6 days after leaf appearance were harvested at 7 days after leaf appearance. For each condition, three biological replicates of three plants each were harvested (FIG. 3C).

Principal component analysis (PCA) separated samples according to their developmental state after leaf four appearance along the first principle component (PC1), while PC2 divided the samples according to treatment. The top 150 most variable genes that positively contributed to PC2 were involved in a multitude of stress responses, whereas the genes that negatively contributed were mainly involved in DNA metabolism and proline catabolism. An increasing amount of up- and downregulated genes were observed in water deficit versus well-watered conditions over time (−0.5>log₂ fold change>0.5 and false discovery rate<0.05), of which only 63 genes were differentially expressed in water deficit conditions over all time points, with 33 upregulated and 30 downregulated genes. The consistently differentially expressed genes in water deficit conditions are mainly associated with inositol biosynthesis, proline and oligosaccharide metabolism, processes that have previously been reported to be water deficit responsive.

To identify the transcriptomic differences corresponding to the different recovery mechanisms, the transcriptomes of rehydration (RW) versus water deficit samples at 5 and 7 days after leaf appearance (RW5 WD5 and RW7_WD7) were compared. The genes specifically upregulated in RW5 plants were mainly enriched for GO categories related to nucleosome and chromatin organization. Both GO categories contain histone genes, including the linker histone H1 and core histones H2A, H2B, H3 and H4. Strikingly, whereas the majority of maize histone genes were upregulated in RW5 plants, two histone genes (GRMZM2G069911 and GRMZM2G086641) showed an opposite expression pattern and were up- and downregulated under water deficit and RW conditions, respectively. Genes specifically downregulated in RW5 samples were involved in glycoside and salicylic acid metabolism. At 7 days after leaf appearance, upregulated genes were involved in trehalose and cellulose biosynthesis, response to oxidative stress and cell wall biogenesis, confirming the increase in mature cell size in RW versus water deficit samples at 7 days after leaf appearance. Downregulated genes were enriched for photosynthesis, response to abiotic stimuli and regulation of transcription. Among those downregulated transcription factors were GRF1/2/3/6/7/8/11/12/13/14/15, AUX/IAA, C2C2-GATA, C2C2-DNA-binding with one finger (DOF), Homeobox, APETALA 2/ETHYLENE RESPONSIVE ELEMENT BINDING LIKE (AP2-EREB), ABA-related transcription factors, TEOSINTE BRANCHED 1/CYCLOIDEA/PCF (TCP) transcription factors, and C2C2-YABBY transcription factors. Moreover, besides the GRFs, also GIF1/AN3 and GIF3 were downregulated at RW7. The GRFs and GIFs showed a higher expression level in RW5 when the division zone is still active compared with the significant downregulation of these transcription factors in RW7, which is specific for growth recovery through cell expansion when the division zone starts to differentiate.

Because the GRFs were specifically differentially expressed at RW7, the role of GRFs in the observed response of maize leaves exposed to drought and rehydration was analyzed.

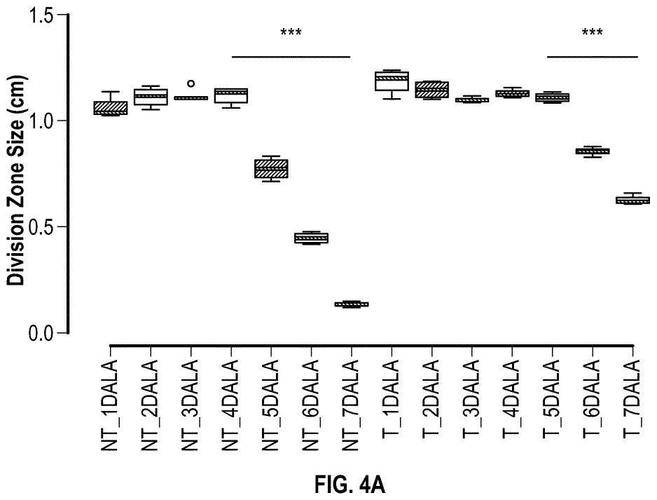

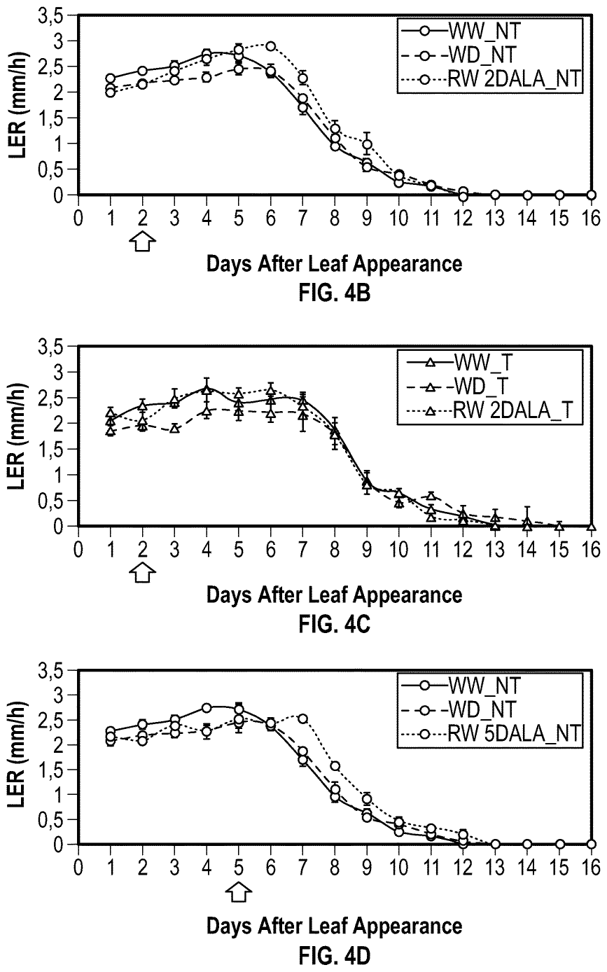

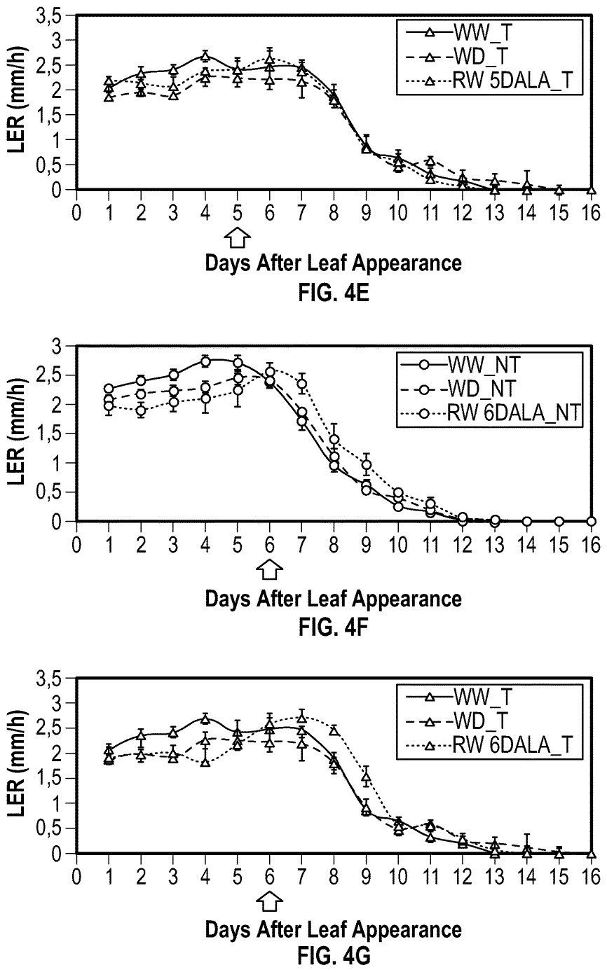

Similar to pGA2ox::PLA1 transgenic plants, the division zone size remained maximal for 1 day more in transgenic GRF1^{R} compared to non-transgenic plants (FIG. 4A), resulting in a significant leaf elongation duration (p_{WW}=0.01 and p_{WD}<0.001) and FLL (p_{WW}=0.01 and p_{WD}=0.04) increase in both well-watered and water deficit conditions. Water deficit-stressed GRF1^{R} transgenic and non-transgenic segregating maize seedlings were rehydrated at 2 to 7 days after leaf appearance. Rehydration of water deficit transgenic and non-transgenic lines at 2 days after leaf appearance up to 4 days after leaf appearance resulted in a full recovery of the leaf elongation rate to that observed under well-watered conditions due to a maximal division zone size in both lines (FIG. 4A-C). The extension of the differentiation of the division zone by one day in water deficit transgenic compared to non-transgenic plants (FIG. 4A) resulted in a full recovery of the leaf elongation rate through cell division in transgenic plants upon rehydration at 5 days after leaf appearance (FIG. 4E). In contrast, non-transgenic plants rehydrated at 5 days after leaf appearance showed a partial recovery of the leaf elongation rate and prolonged leaf elongation duration in comparison to water deficit conditions and thus a growth recovery through cell expansion (FIG. 4D). Rehydration at later timepoints in both lines resulted in a partial leaf elongation rate recovery and prolonged leaf elongation duration through cell expansion due to the differentiation of the division zone (FIG. 4A, FIG. 4F, and FIG. 4G).

Consequently, the prolongation of growth recovery through cell division in the transgenic plants upon rehydration was also shown by a significant difference in leaf elongation duration between transgenic and non-transgenic plants upon rehydration at 6 days after leaf appearance (p=0.05) but not at 5 days after leaf appearance (p=0.59) (FIG. 4D-G).

These data show that growth regulators that were previously assumed to be associated with cell division, like GRF1, are also involved in the maintenance of cell division activity, which is important for growth recovery upon rehydration. In addition, the RNA-seq time series of water deficit and RW conditions allowed the identification of potential new key players in the maintenance of cell division activity.

### Example 4: Multiplex Genome Editing Reveals Novel Molecular Players Involved in Drought Tolerance

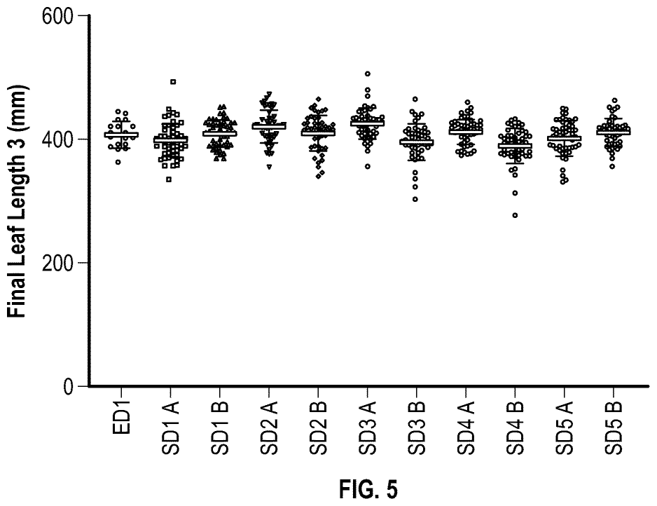

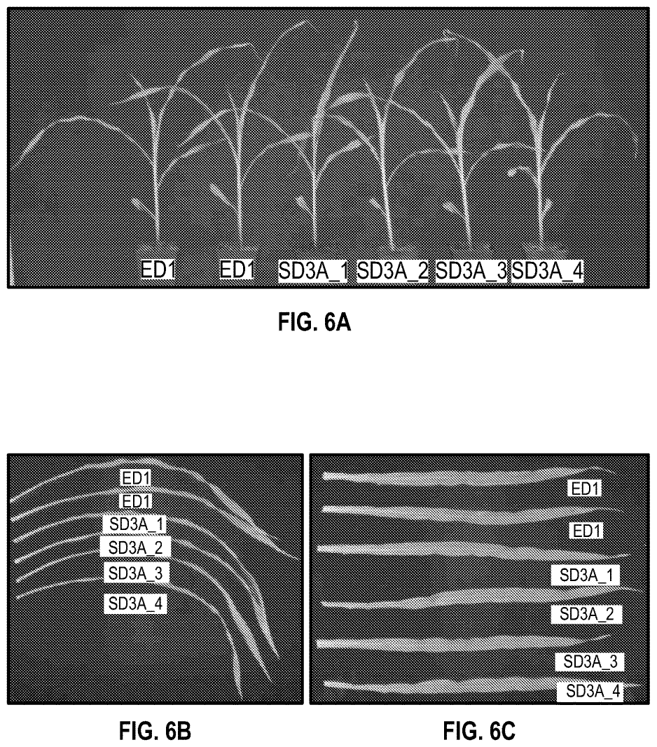

To validate the potential of some of the genes that were co-expressed with the GRFs, 40 genes that were upregulated under water deficit conditions and downregulated during rehydration (marked as REW in Table 2) were selected for multiplex genome editing using the BREEDIT approach (Lorenzo et al. (2023) The Plant Cell 35:218-238. doi: 10.1093/plcell/koac243). In addition, 11 genes that have previously been described to be water deficit response genes in literature (marked as POS in Table 2) and 9 genes unrelated to water deficit response (marked as NEG in Table 2) were included as positive and negative controls, respectively. Table 2 summarizes the genes selected for multiplex CRISPR/Cas9 editing. In the BREEDIT approach, SCRIPT constructs each expressing twelve gRNAs are transformed in a Cas9 expressing B104 maize line, further referred as EDITOR. The obtained TO plants are then used for further breeding. Subsequently, phenotyping and multiplex amplicon sequencing of all selected genes allows for correlating traits with causative gene combinations. In total, five scripts, targeting 60 genes were constructed and denominated Script Drought 1 (SD1) to SD5 (Table 2). The different scripts were used to transform the EDITOR, resulting in transgenic TO lines that contain both Cas9 and the scripts. For each script family, the TOs lines were both selfed and backcrossed with B104 and further progenies were used for phenotyping experiments under moderate drought conditions. A total of ten populations were phenotyped: two T2 lines derived from a selfed backcross with B104 for SD1 and 2, and two T1 lines derived from independent selfed TOs for SD3, 4 and 5, using the final length of the third leaf (FLL3) as a readout for growth under water deficit conditions (FIG. 5). Out of the ten, four populations were identified (SD2A, SD3A, SD4A and SD5B) that on average displayed an increased FLL3 than that of the control EDITOR, SD3A being the most prominent one amongst them (FIG. 6A-C).

TABLE 2

| | | Script | | |
|---|---|---|---|---|
| Gene | Description | number | gRNA_UID | Class |
| GRMZM2G179325 | gras3 - GRAS-transcription factor 3 | 1 | 101 | NEG |
| GRMZM2G041223 | Putative growth-regulating factor 6 | 1 | 102 | REW |
| GRMZM2G099862 | Growth-regulating factor 2 | 1 | 103 | REW |
| GRMZM2G102770 | ZmCLE10B/FOS1/FON2 SPARE1 | 1 | 104 | NEG |
| GRMZM2G133675 | bHLH187 | 1 | 105 | REW |
| GRMZM2G167824 | yabby10 | 1 | 106 | REW |
| GRMZM2G479760 | BZIP4/ABF family | 1 | 107 | REW |
| GRMZM2G079200 | iaa26 - Aux/IAA-transcription factor 26 | 1 | 108 | REW |
| GRMZM2G025685 | Heat stress transcription factor 27 | 1 | 109 | REW |
| GRMZM2G058404 | ggps3 - geranylgeranyl pyrophosphate synthase3 | 1 | 110 | NEG |
| GRMZM2G135381 | gata32 - C2C2-GATA-transcription factor 32 | 1 | 111 | REW |
| GRMZM2G158140 | ARR16 | 1 | 112 | REW |
| GRMZM2G059102 | MADS-transcription factor 68 | 2 | 201 | POS |
| GRMZM2G041127 | hb54 - Homeobox-transcription factor 54 | 2 | 202 | POS |
| GRMZM2G163015 | ZmACS1 | 2 | 203 | POS |
| GRMZM2G085751 | bhlh24 - bHLH-transcription factor 24 | 2 | 204 | REW |
| GRMZM2G105348 | hsftf18 - HSF-transcription factor 18 | 2 | 205 | REW |
| GRMZM2G012724 | wrky83 - WRKY-transcription factor 83 | 2 | 206 | NEG |
| GRMZM2G056369 | icl1 - isocitrate lyase1 | 2 | 207 | NEG |
| GRMZM2G005732 | cct43 - CO CO-LIKE TIMING OF CAB1 protein | 2 | 208 | REW |
| | domain43 | | | |
| GRMZM2G359924 | iaa37 - Aux/IAA-transcription factor 37 | 2 | 209 | REW |
| GRMZM2G139160 | MRP interacting1 | 2 | 210 | POS |
| GRMZM2G119359 | GRF12 | 2 | 211 | REW |
| GRMZM2G145085 | transcription initiation factor IIF subunit alpha RAP74 | 2 | 212 | REW |
| GRMZM2G074393 | wiskott-aldrich syndrome family protein, putative | 3 | 301 | REW |
| | (DUF1118) | | | |
| GRMZM2G109879 | ABI3-VP1-transcription factor 28 | 3 | 302 | REW |
| GRMZM2G002915 | hb34 - Homeobox-transcription factor 34 | 3 | 303 | REW |
| GRMZM2G077197 | traf21 - TNF receptor-associated factor 21 | 3 | 304 | REW |
| GRMZM2G068330 | zhd1 - ZF-HD-transcription factor 1 | 3 | 305 | REW |
| GRMZM2G089132 | ubiquinone biosynthesis protein (Protein of unknown | 3 | 306 | REW |
| | function, DUF547) | | | |
| GRMZM2G161435 | hb2 - Homeobox-transcription factor 2 | 3 | 307 | REW |
| GRMZM2G133631 | ZmPYL9 - pyrabactin resistance-like protein 9 | 3 | 308 | POS |
| GRMZM2G045678 | dof39 - C2C2-Dof-transcription factor 39 | 3 | 309 | REW |
| GRMZM2G081892 | AP2-EREBP-transcription factor 118 | 3 | 310 | REW |
| GRMZM5G832994 | rtl1 - reversion-to-ethylene sensitivity1 like1 | 3 | 311 | NEG |
| GRMZM2G001799 | iaa41 - Aux/IAA-transcription factor 41 | 3 | 312 | REW |
| GRMZM2G163761 | Knotted1-interacting protein | 4 | 401 | POS |
| GRMZM2G480903 | grx2 - glutaredoxin2 | 4 | 402 | REW |
| GRMZM2G062244 | hb107 - Homeobox-transcription factor 107 | 4 | 403 | REW |
| GRMZM2G079727 | mads36 - MADS-transcription factor 36 | 4 | 404 | REW |
| GRMZM2G059544 | iaa16 - Aux/IAA-transcription factor 16 | 4 | 405 | REW |
| GRMZM2G053298 | nlp13 - NLP-transcription factor 13 | 4 | 406 | REW |
| GRMZM2G154987 | ZmPYL3 - pyrabactin resistance-like protein 3 | 4 | 407 | POS |
| GRMZM2G305856 | myb21 - MYB-transcription factor 21 | 4 | 408 | NEG |
| GRMZM2G177386 | ZmPP2C4 (clade A) | 4 | 409 | POS |
| GRMZM2G151542 | ereb80 - AP2-EREBP-transcription factor 80 | 4 | 410 | REW |
| GRMZM2G118047 | hsftf28 - HSF-transcription factor 28 | 4 | 411 | REW |
| GRMZM2G086949 | arftf29 - ARF-transcription factor 29 | 4 | 412 | REW |
| GRMZM2G054361 | ZmACS6 - 1-aminocyclopropane-1-carboxylate | 5 | 501 | POS |
| | synthase6 | | | |
| GRMZM2G033962 | Two-component response regulator-like APRR3 | 5 | 502 | REW |
| GRMZM2G129777 | ereb192 - AP2-EREBP-transcription factor 192 | 5 | 503 | REW |
| GRMZM2G048733 | ZmPYL11 - pyrabactin resistance-like protein 11 | 5 | 504 | POS |
| GRMZM2G473711 | gla3 - beta-glucanase3 | 5 | 505 | NEG |
| GRMZM2G031983 | gata15 - C2C2-GATA-transcription factor 15 | 5 | 506 | REW |
| GRMZM2G098594 | Growth-regulating factor 14 | 5 | 507 | REW |
| GRMZM2G011932 | gbfl - G-box binding factor | 5 | 508 | POS |
| GRMZM2G449681 | wrky92 - WRKY-transcription factor 92 | 5 | 509 | NEG |
| GRMZM2G138967 | C2C2-GATA-transcription factor 17 | 5 | 510 | REW |
| GRMZM2G033413 | bzip100 - bZIP-transcription factor 100 | 5 | 511 | REW |
| GRMZM2G057753 | ofp38 - OVATE-transcription factor 38 | 5 | 512 | REW |

These top 4 performing populations were then tested in a phenotyping experiment under water deficit conditions in the PHENOVISION automated greenhouse system (Verbraeken et al. (2021). Plant Physiol. 186:336-1353). Simultaneously, all plants were genotyped to analyze their edit profiles.

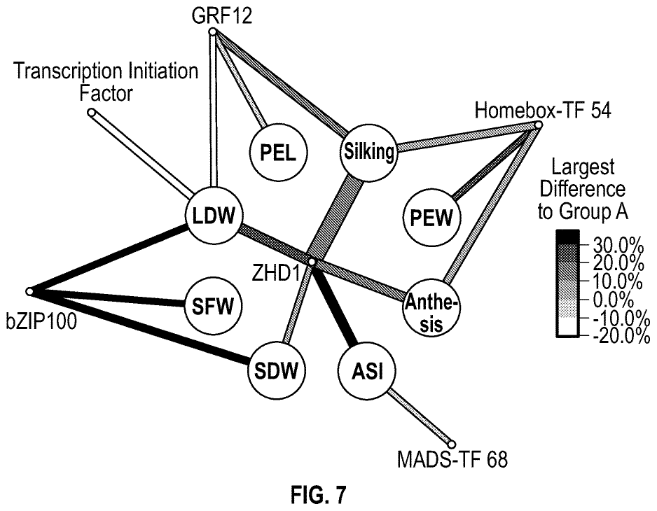

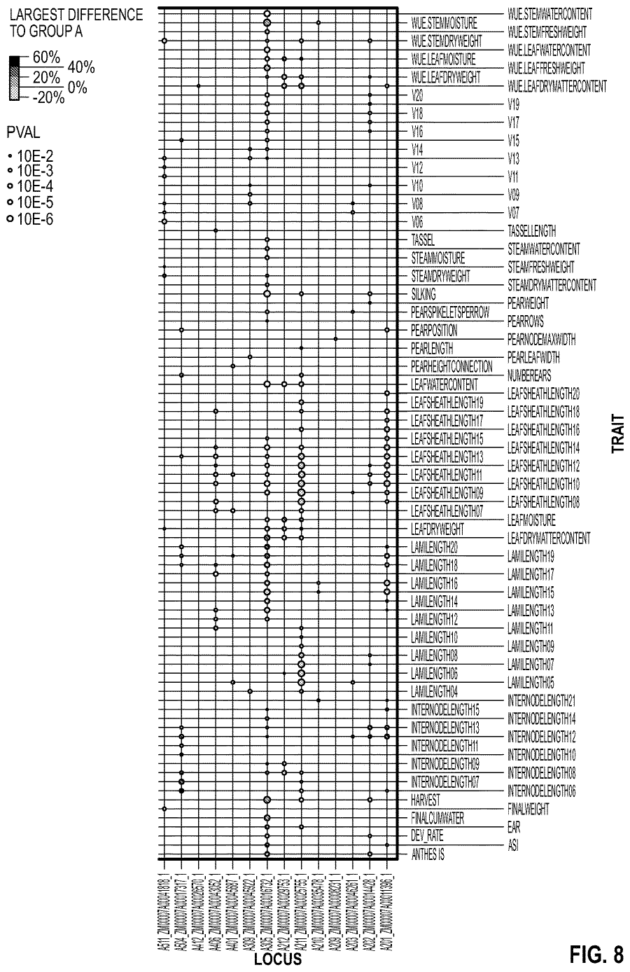

On the genotype level, 40 edited loci were detected out of 48 targeted for all four script populations. Out of these, 19 loci (gRNAs 201, 202, 203, 204, 205, 206, 209, 210, 211, 212 from SD2; 305, 309 and 311 from SD3; 401, 406 and 412 from SD4 and 504, 505 and 511 from SD5) presented sufficient segregation data to be analyzed in phenotype-to-genotype associations. For the associations, agronomically relevant phenotypes such as biomass, ear parameters, flowering and the anthesis-silking interval (ASI) were focused on (FIG. 7), but phenotypical data on other developmental traits were also included in the association studies (FIG. 8). Table 3 shows the number of significant genotype-phenotype associations found in the different gene groups of SD 2 to SD5. Some genes could not be associated because of the homozygous nature of their edits. In the POS subgroup, all genes had a significant association with one of the traits, whereas none of the edits in the NEG subgroup associated with any of the traits phenotyped under water deficit conditions (Table 3), showing the power of the gene selection and screening method. In the REW subgroup, 80% of significant single genotype-phenotype associations were detected for targeted genes of which associations were possible (Table 3).

TABLE 3

| | Genes in SD2 | Associations | Significant | |
|---|---|---|---|---|
| Gene groups | to SD5 | possible | associations | Respective gRNA_UID |
| POS genes | 11 | 6 | 6 | 201; 202; 203; 210; 401; 504 |
| REW genes | 31 | 10 | 8 | 209; 211; 212; 305; 309; 406; 412; 511 |
| NEG genes | 6 | 3 | 0 | |
| Total amount | 48 | 19 | 14 | |
| of genes | | | | |

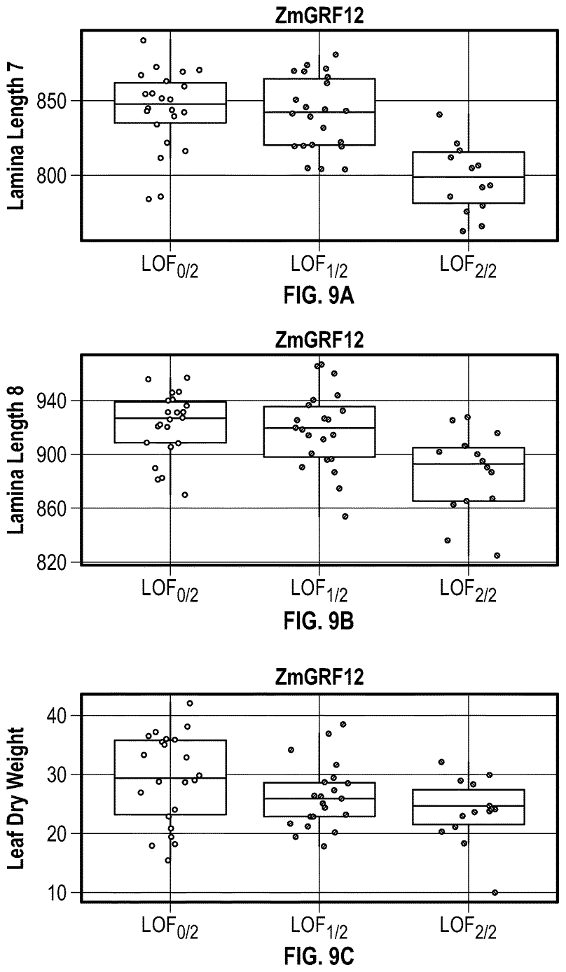

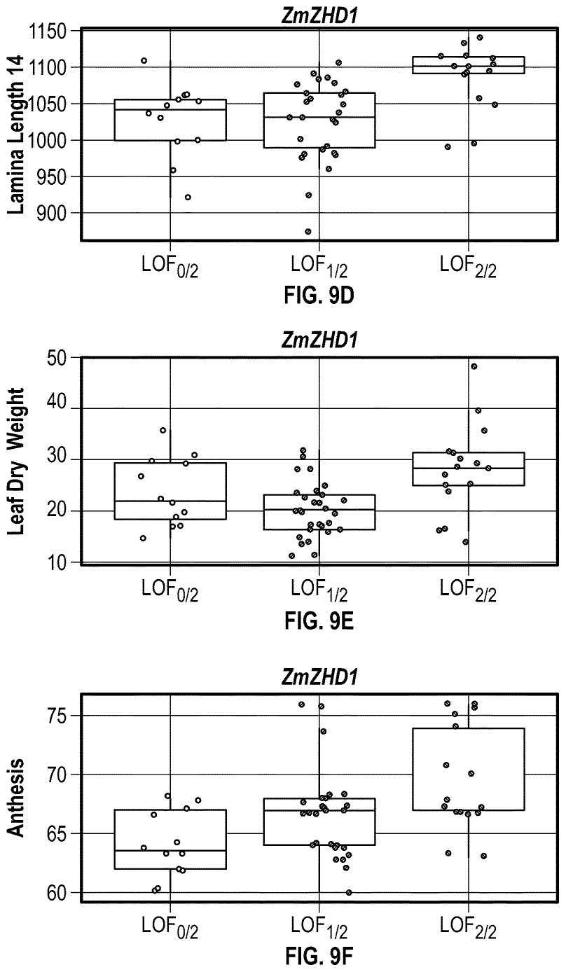

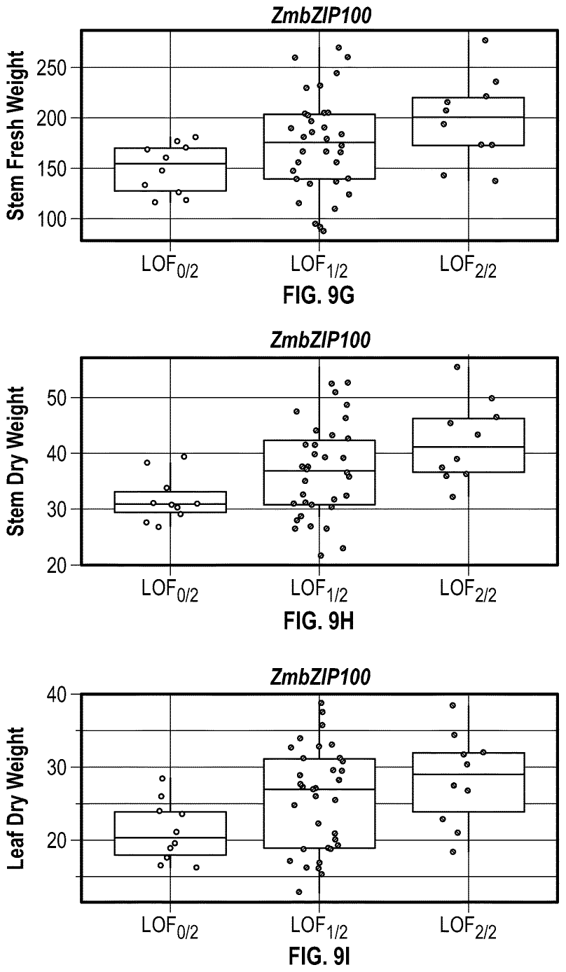

For five out of six genes from the POS subgroup, significant positive effects were observed under water deficit conditions (FIG. 8; Table 3). Edits in the novel REW genes associated with both positive and negative changes in several developmental and agronomical traits. Edits in ZmGRF12 (gRNA 211, GRMZM2G119359) led to a decrease in early internode length (internodes number 6, 7 and 8 specifically). It also linked with cumulative reductions in leaf lamina length (leaves 4 to 9; FIG. 8, FIG. 9A-B), decreases in leaf dry weight (FIG. 7, FIG. 9C) and leaf dry matter content and primary ear length (FIG. 7, FIG. 8). Interestingly, while leaf lamina values were negatively affected, leaf sheath values were significantly increased in plants edited on ZmGRF12 (FIG. 8, leaves 7 to 14, 16, 18 and 19). Edits in GRMZM2G145085 (gRNA 212), encoding a transcription initiation factor subunit alpha, resulted in significant decreases in leaf dry weight and leaf dry matter content (FIG. 7, FIG. 8). Edits in ZF-HD-TRANSCRIPTIONFACTOR1, ZmZHD1 (gRNA 305, GRMZM2G068330) led to significant increases in lamina leaf lengths (FIG. 9D), leaf dry weight (FIG. 7, FIG. 9E) and leaf dry matter content and decreases in internode length (FIG. 8). However, a delay in development was seen for ZmZHD1 edited lines, observed as increases in days to reach V stages, delayed anthesis and an increased ASI (FIG. 7, FIG. 8, FIG. 9F). Edits in GRMZM2G045678 (gRNA 309), encoding a C2C2-Dof-transcription factor, increased the primary ear leaf width while targeting a NIN-LIKE PROTEIN (NLP) transcription factor (gRNA406, GRMZM2G053298) had a negative effect on leaf sheath and lamina length (FIG. 8). Editing GRMZM2G033413 (gRNA 511), encoding a bZIP transcription factor, led to a significantly increased stem and leaf dry weight and stem fresh weight (FIG. 7, FIG. 8, FIG. 9G-I). The multiplex BREEDIT approach showed that edited genes upregulated under water deficit conditions and downregulated during rehydration give rise to a variety of growth related phenotypes.

What is claimed is:

1. A maize plant comprising a loss-of-function allele of the endogenous maize gene of SEQ ID NO: 1, 3, 5, 7, 9, and/or 11, or an allelic variant thereof.

2. The maize plant of claim 1, wherein the loss-of-function allele comprises an amorphic allele of the endogenous maize gene.

3. The maize plant of claim 1, wherein the loss-of-function allele comprises a hypomorphic allele of the endogenous maize gene.

4. The maize plant of claim 1, wherein the plant is homozygous for the loss-of-function allele of the endogenous maize gene.

5. The maize plant of claim 1, wherein the loss-of-function allele comprises a frameshift mutation, a missense mutation, or a nonsense mutation in the endogenous maize gene.

6. The maize plant of claim 1, wherein the plant comprises a loss-of-function allele of the endogenous maize gene of SEQ ID NO: 1, or an allelic variant thereof.

7. The maize plant of claim 1, wherein the plant comprises a loss-of-function allele of the endogenous maize gene of SEQ ID NO: 3, or an allelic variant thereof.

8. The maize plant of claim 1, wherein the plant comprises a loss-of-function allele of the endogenous maize gene of SEQ ID NO: 5, or an allelic variant thereof.

9. The maize plant of claim 1, wherein the plant comprises a loss-of-function allele of the endogenous maize gene of SEQ ID NO: 7, or an allelic variant thereof.

10. The maize plant of claim 1, wherein the plant comprises a loss-of-function allele of the endogenous maize gene of SEQ ID NO: 9, or an allelic variant thereof.

11. The maize plant of claim 1, wherein the plant comprises a loss-of-function allele of the endogenous maize gene of SEQ ID NO: 11, or an allelic variant thereof.

12. The maize plant of claim 1, wherein water use efficiency of the maize plant is increased in comparison to water use efficiency of a control maize plant lacking the loss-of-function allele.

13. The maize plant of claim 1, wherein drought tolerance of the maize plant is increased in comparison to drought tolerance of a control maize plant lacking the loss-of-function allele.

14. The maize plant of claim 1, wherein internode length, leaf lamina length, leaf sheath length, leaf dry weight, leaf dry matter content, and/or primary ear length are decreased in comparison to internode length, leaf lamina length, leaf sheath length, leaf dry weight, leaf dry matter content, and/or primary ear length for a control maize plant lacking the loss-of-function allele.

15. The maize plant of claim 14, wherein internode length, leaf lamina length, leaf sheath length, leaf dry weight, leaf dry matter content, and/or primary ear length are decreased under drought conditions.

16. The maize plant of claim 1, wherein stem fresh weight, stem dry weight, leaf dry weight, leaf sheath length, leaf lamina length, and/or primary ear leaf width are increased in comparison to stem fresh weight, stem dry weight, leaf dry weight, leaf sheath length, leaf lamina length, and/or primary ear leaf width for a control maize plant lacking the loss-of-function allele.

17. The maize plant of claim 16, wherein stem fresh weight, stem dry weight, leaf dry weight, leaf sheath length, leaf lamina length, and/or primary ear leaf width are increased under drought conditions.

18. The maize plant of claim 1, with the proviso that the plant is not exclusively produced by an essentially biological method.

19. The maize plant of claim 1, wherein the plant comprises elite germplasm.

20. The maize plant of claim 1, wherein the plant further comprises: (i) one or more mutations in a distinct maize gene; and/or (ii) one or more transgenes, optionally wherein the transgene encodes a protein or RNA conferring herbicide tolerance or pest tolerance.

21. A maize plant part of the plant of any one of claims 1-20, wherein the maize plant part comprises the loss-of-function allele.

22. The maize plant part of claim 21, wherein the part is a seed, stalk, stem, pollen, or leaf.

23. The maize plant part of claim 21, wherein the part is a seed.

24. The maize plant part of claim 23, wherein the seed further comprises at least a partial coating of a composition comprising a biological agent, nematicide, insecticide, or fungicide.

25. A maize plant cell of the plant of any one of claims 1-20.

26. A method of producing maize seed, comprising growing the maize plant of any one of claims 1-20 and harvesting seed therefrom.

27. A biological sample comprising a nucleic acid containing a loss-of-function allele of the endogenous maize gene of SEQ ID NO: 1, 3, 5, 7, 9, and/or 11, or an allelic variant thereof.

28. The biological sample of claim 27, wherein the biological sample comprises material obtained from the maize plant of any one of claims 1-20 or a part thereof, optionally wherein the part is a seed.

29. The biological sample of claim 27, wherein the biological sample is non-regenerable.

30. The biological sample of claim 27, wherein the biological sample comprises maize seed meal.

31. A method of producing a commodity maize plant product, the method comprising: (i) processing the maize plant of any one of claims 1-20 or a maize seed obtained therefrom; and
(ii) recovering the commodity maize plant product from the processed maize plant or maize seed.

32. The method of claim 31, wherein the commodity maize plant product is seed meal, starch, syrup, silage, oil, or protein.

33. The method of claim 31, wherein the commodity maize plant product comprises a detectable amount of a DNA molecule comprising the loss-of-function allele of the endogenous maize gene.

34. A guide RNA molecule comprising a spacer RNA molecule that targets the endogenous maize gene of SEQ ID NO: 1, 3, 5, 7, 9, 11, or an allelic variant thereof.

35. The guide RNA molecule of claim 34, wherein the spacer RNA molecule comprises the RNA encoded by SEQ ID NO: 13, 14, 15, 16, 17, or 18.

36. A method for generating the maize plant of any one of claims 1-20 comprising introducing a loss-of-function allele in the endogenous maize gene of SEQ ID NO: 1, 3, 5, 7, 9, and/or 11, or an allelic variant thereof.

37. The method of claim 36, wherein the loss-of-function allele is introduced by:
(i) directing both: (a) a guide RNA (gRNA) molecule comprising a spacer RNA molecule that targets the endogenous maize gene of SEQ ID NO: 1, 3, 5, 7, 9, and/or 11, or an allelic variant thereof, and (b) an RNA dependent endonuclease (RDE) that recognizes the gRNA molecule to the genome of a target maize plant cell; and
(ii) isolating a maize plant comprising the loss-of-function allele from the maize plant cell.

38. The method of claim 36, wherein the directing of the gRNA and the RDE to the genome of the target maize plant cell comprises introducing the gRNA, the RDE, a gRNA/RDE complex, a nucleic acid encoding the gRNA, and/or a nucleic acid encoding the RDE into the target maize plant cell.

39. The method of claim 36, wherein the spacer RNA molecule comprises the RNA encoded by SEQ ID NO: 13, 14, 15, 16, 17, or 18.

40. The method of claim 36, wherein water use efficiency of the maize plant is increased in comparison to water use efficiency of a control maize plant lacking the loss-of-function allele.

41. The method of claim 36, wherein drought tolerance of the maize plant is increased in comparison to drought tolerance of a control maize plant lacking the loss-of-function allele.

42. The method of claim 36, wherein internode length, leaf lamina length, leaf sheath length, leaf dry weight, leaf dry matter content, and/or primary ear length are decreased in comparison to internode length, leaf lamina length, leaf sheath length, leaf dry weight, leaf dry matter content, and/or primary ear length for a control maize plant lacking the loss-of-function allele.

43. The method of claim 42, wherein internode length, leaf lamina length, leaf sheath length, leaf dry weight, leaf dry matter content, and/or primary ear length are decreased under drought conditions.

44. The method of claim 36, wherein stem fresh weight, stem dry weight, leaf dry weight, leaf sheath length, leaf lamina length, and/or primary ear leaf width are increased in comparison to stem fresh weight, stem dry weight, leaf dry weight, leaf sheath length, leaf lamina length, and/or primary ear leaf width for a control maize plant lacking the loss-of-function allele.

45. The method of claim 44, wherein stem fresh weight, stem dry weight, leaf dry weight, leaf sheath length, leaf lamina length, and/or primary ear leaf width are increased under drought conditions.

46. The method of claim 36, wherein the loss-of-function allele is introduced by crossing a maize plant comprising the loss-of-function allele with a second maize plant and harvesting F1 seed comprising the loss-of-function allele, thereby producing progeny maize seed comprising the loss-of-function allele.

47. A method for determining whether a maize plant cell, plant part, plant, or biological sample obtained therefrom comprises a loss-of-function allele of the endogenous maize gene of SEQ ID NO: 1, 3, 5, 7, 9, and/or 11, or an allelic variant thereof, the method comprising:
(i) analyzing a polynucleotide comprising a portion of SEQ ID NO: 1, 3, 5, 7, 9, and/or 11, or an allelic variant thereof, or analyzing an RNA encoded by a portion of SEQ ID NO: 1, 3, 5, 7, 9, and/or 11, or an allelic variant thereof, from the maize plant cell, plant part, plant, or biological sample, wherein an insertion, deletion, and/or substitution of one or more nucleotides in the polynucleotide or RNA is indicative of the presence of the loss-of-function allele in the maize plant cell, plant part, plant, or biological sample; and/or
(ii) analyzing a polypeptide encoded by SEQ ID NO: 1, 3, 5, 7, 9, and/or 11, a portion thereof, or an allelic variant thereof from the maize plant cell, plant part, or plant, wherein an insertion, deletion, and/or substitution of one or more amino acid residues of the polypeptide or a change in the biologic or biochemical activity of the polypeptide is indicative of the presence of the loss-of-function allele in the maize plant cell, plant part, plant, or biological sample.


SEQUENCE LISTING (18 sequences)

SEQ ID NO: 1 - DNA, 3016 bp, Zea mays
  source 1..3016: mol_type = genomic DNA
acccccacctgaccttctcttcctccctcccccgaccgcctgtctgtcctccgcgcatccgttctctatcgaaagggaggaggaggagcgcgcgggagtgggctgggggcccaccgatgctgagctcggcgtcctcggccggggcggccatggggatgggcggcgggtaccaacaccagccgctgccactgccgcagcgcggggcggcggccgcggtcttcaccgccgcgcagtgggcggagctggagcagcaggcgctcatctacaagtacctcatggccggcgtccccgtcccgcccgatctcctccgccccgccccccacgccgccgccttctccttcgccagccccgccgcgtcgcccttctaccatcaccaccaccaccacccgtcccgtaagctccccccttacctccatccccctctttttttttgttcgggagagaacagttgcatagatcttttgcccgccatgcttcttactgggattggatttgtctggttgggtattgccgcgatttatctcatcgagtgattcattaagcatgcgctgcctagcatctcggcatctgcctaatgttcgagtggggtggatctttgtcacctcctcacccgcttgctcgcgcaatgggatggggattctttattattcaaacttttaaacaaaacaaagtgggagaaagcatcttcgcattgggttggtcaaatgttcatgtgctttttgatctgccccagtagcaagtacaccatttcattataatatatgtatatactatggtttgtttcccttctgcagtgagttactacgcctactacgggaagaagctggacccggagccgtggcggtgccgccgcaccgacggcaagaagtggcggtgctccaaggaggcgcaccccgactccaagtactgcgagcgccacatgcaccgtggccgcaaccgttcaagaaagcctgtggaatccaagaccgcctcctcgccgccccagctgtccaccgtcgtcaccaccaccaccacccgggaggccgccgccgcgacgccccttgagtccctcgcgggggcggggggtaaggctcacggcctgtccctcggcggcggggctggctcgtcgcacctcagcgtcgacgcttcgaacgctcactttcgctatggcagcaagtatggttacattcgcagttcccttttttgtggttctagctgttgttgctatcgaatggccatgatgctatagttttcgttggttctacactattatcgcaaaaggagaatcacaccgactcactcactcttcaacggcaaatgcacttctgccactggcttcttgcagtattagcccttctactgtacaatatagaaatcttttgcagttctgttgtattaagtgaactgccgtgcctggtatgccctgggccctggctgcttctgctatcttagatgtctataaaattttatatatttgcatgctctgaacaacttgggctggcggatgcccatgtccatgccgctacacaacttaggtctgtactagtgtttcaaacagattttcttattggccccctattttgtactccgtttctttcggattcattgtttttgtgggtttgaatttctataaggatttgcgcatcaaatattgatatgacttcaagacaatattgcagttcatgtcctgaatttgcatcctgagcttgattttctgtcgagttgactttaatagctatttatttgtcacttgtacatctattccacttcataccaaacaaacccaccatgtcttgccggttcctgtaggcagaaatatgacactagatagactccaaagctatagtgccaggctgacaagtgttctgtcagttcataatactttcgttggcaacttgtgattgtcacatactatccataactaattgactgtgcaggtaccctcttggagctaaatccgatgctggcgagctgagcttcttctcaggagcaccagggaactccaggggcttcaccattgattctccagcagataactcttggcactccctgccatccaacgtgcccccgtttacactgtccaagggcagagactctggcctcctgcccggtgcctactcctactcccacattgaaccaccgcaggtgctcggccaggtcaccatcgcctcgctgtcacaggaccaggagcgccaccagtcgtcgttcagcagcggcggggccggcgctggtggattgctggggagcgtgaagcaggagaaccagccgctgaggcccttcttcgacgagtggcctgggacgcgggactcgtggtcggagatggacgacgcgaggtccagtaggacctccttctcgacgacccagctctccatctccattccgatgcccagatgtgagttgtttcatactgatctcatcttaggaacccgcagtccttctgtgtgcttccccaatcgtgcttggatgtgttctgatgacagcagcaatcacaattgtttctgcaggtgattgagaacgaagctcgtgcgcggccagtaggtggcgttgtcgcatgacgcgcacccagcatcgcgctcgctgctagcgagccaccgtcacccggtgattgatgccctcatcctagctatttgtttcgctgaatcgcattaagaacagaactctatgtaagagcaggatccttcctcatgtatcaatgggggcaatgaactccattctgtttctatggttgtagtaccgaccatgctttgtatttgttgtacccagaccgagaatgaaaaccgcccagtcgtcaggcgttgtcgtcgctgcatcagatgatccaagtgtattgtgtgctttccaggctgtgtaagatctgatggcgaatctgttactgcagtgtaggtgtgtagcatctgtggtatggtaccttttgttgtgctactggatctggattgatgcaaaatctggtgctgccgtaggcccctcttccttttctctactgctataataataatacatcttttttttatgataggaaaagaaatgagaagaacag

SEQ ID NO: 2 - protein, 313 aa, Zea mays
MAGVPVPPDLLRPAPHAAAFSFASPAASPFYHHHHHHPSLSYYAYYGKKLDPEPWRCRRTDGKKWRCSKEAHPDSKYCERHMHRGRNRSRKPVESKTASSPPQLSTVVTTTTTREAAAATPLESLAGAGGKAHGLSLGGGAGSSHLSVDASNAHFRYGSKYPLGAKSDAGELSFFSGAPGNSRGFTIDSPADNSWHSLPSNVPPFTLSKGRDSGLLPGAYSYSHIEPPQVLGQVTIASLSQDQERHQSSFSSGGAGAGGLLGSVKQENQPLRPFFDEWPGTRDSWSEMDDARSSRTSFSTTQLSISIPMPRCD

SEQ ID NO: 3 - DNA, 5574 bp, Zea mays
  source 1..5574: mol_type = genomic DNA
ttgccgttttcgaccccagccgttaaaagcgccacgcgtgacgcgcccacgccgccttccacatggcctcaaaccaaacgaaccaaaccctacgtagcgagcacaccgcgggcggcggcataagcatggggagcgtcgcgcccgccgaactggtcctgaaggcggcgtgcgagcgctgcggtgcagcgtcgggactctacgcgacgtcgtgccggcacaccacgctctgctccgactgtggcatgaccttggcgctcgcccgcgggcgttgcaccgtgtgcagcgcccccgttaccaggctcatccgggtacggtacggccccgcgccgtctctaatgcgagatatcatctaatgcagttcctcgttggcaatcgggcgagcaatgcagatgcagctatctgcgtatcgtttgttcccccggtgatttctcgttattcacaccgctcgctctgcaggagtacgatgtccgggtggatacctccgcggagaaggagaagacccactacatcggcaggttcaccaccggcttgccgcccttatcgaatgagaggagcgcagggaatagatggactcttcgcaaggatgtcccccatggacagcagcttactgggaatatgcgggtactggcttgatgatcatagttgccattcttttgctttggacgacctgacaattctgtcacccaattagtgtccttaaccatacatagaaaacagtgttacaatggtgaacctgagtaaattctaattatccacaatcaatctaaataacacaatagttactgcaaagtagttatgctttatcagacatctcgtatcagttgcaaattggcagtatgcagttgacaaatatgtattgctagaagatatctgttcaatagcaacttacggacaataagctgatgtttcccctgttaaaccatgtgcacgcatttgataaaacaggacaagcactacagcaagatgccatggattctggaggatgagacgggggaacatcagtatcagggccaaactgaggatccacctgccacatactattcgcttacgcaaaaaggcaatgacatgattgctgttcaagttggttcctggtactatactctctgcttccctctgttgttattagtcttcaaatgaatatatccatcatctgaacatacatgcttgcaagattcaagtacgttaaattgtatatatttcatcaagtcttggcattactaattgtgtgtcagtaagctcctagtataatttcttttgagatgagtgctctcttaattttgttagagcgttcttacagtctctcatgccctatcaggtacaacttcagtaaaattgcacagtacaaacagctgaccttggaagaagctgaagagaaaatgaatagaaggagaagcagtgcatctgggtatgaacgatggatgatgaaggcagctacaaatggacctgatgcctttagttcagttgtgaagaggcttgatgatgtaaatagaggggcaacaaacggtgttcaccccaagaaaggagataggaatgagaatggaaatcaatctgacaaagttgatgacaaagaagaagagggagctgcaaggaaaaacaggcttgggcttactatgaaggacatgggcgaggattatgaggatagtggaaacgacatagaatttgatttggatgatgaaattgagaaaggtgtatacatatcaaatactacccggaagaatgagtattacttttgctaatactgagatttcatgaatttctgcatgcactataaggcacatattttattgttattcctcagaactagtcatagtttcacacctggaagaatgaaaagactgtatgccatgtttccatacccatattctcggacttcaatccatcatgtatcttgttgagtttgtgatctaaattctgcatatggaggccctgcaagtctaggggtgcgggtgtcatggaccttcggtccatggggatagctgtcttctccggcgaggttatacccctctgccgcaggcttggttctagggtagcagccctaggcgcttgagagtcattgcaagagagagagattggtttgataatatattcctttcccctatgtctcttgcgtacaggcctatatataggccaccggttgaccaactaggcaaccattaattaggcaagtgactaattgaaattatctcccctacaatttaggtctatcaatttagccaccaaatcagtcactttcctttgccatgccttccttgcacgcgctttcctttgccatgccttccttgtgcgcgctttcctttgccatgctttccttgcgcgcgctgctgccagcgcccgtgccttccttgcacgcgctgctgccagcgcccctgcggcctctcgcttttggcgcgcataggtgcggccccacatgagtgaccgctccctatgatgtccatgacatctcctcctcccttgaggaccagctcgtcctcgagctgccatagacttacctgacacgacttaaggttaagccttttacatctcggcttacctttattatttaagacgaaaatacaaaataattgtggaactaaaatataaatttgaactgcaactatgtcctcctgtcctcctggatcccaaggaaagagccgaactgcggccttcacgttggatgaaaggtggaggaggaaccagcccgttcttatgttgggtctgtccagcaccaagacaggtgcccgaatgaaggagacaaccctctttggccgtgcagtgggactgcatacccagatctcgacttctgcagggggttcaaaaagtatagacgagacctggtggttgggcgcgcaggctgcgagttggtgcagcgatgagctgatcagtaagtgcagcttccgcaccgcccgcttaacaaggaagtcgcgcactgcggcttgcaggcgcactgcggcttgcaagcgcaccacggcctgctcatgtgatgacggccatggggtggccctggagggcacaacaaggtgcttctccccacgaagggactgtacctggcggcgtgccaggaaccctcgcgctgccgtctgaagccgcaccgctgcttggagttccttctctgtcttctccttgtagcggtggaacatcacaacgaactgatcccactgctctgcgaagcggccaaaactctctttgagccgagtgaatgcatctggagttggagagggcgggtgggagggcgttgcggcggctgatggcgcggcagctggtggtggtgagggatgggcggctggtggtggtgaggataacctggagctgataccaggtgtcatggaccttcggtccatgaggatagctgtcttctccggcgaggttatacccctctgccgcaggcttggttctagggtagcagccctaggcgcttgagagtcattgcaagagagagagattggtttgataatatattcctttcccctatgtctcctgcgtacaggcctatatataggccaccggttgaccaactaggcaaccattaattaggcaagtgactaattgaaattatctcccctacaatttaggtctatcaatttagccaccaaatcagtcactttcctttgccatgccttccttgcgcgcgctttcctttgccatgccttccttgtgcgcgctttcctttgccatgccttccttgcgcgcgctgctgccagcgctcgtgccttccttgcacgcgctgctgccagcgccccctgcggcctctcgcttttggcgcgcataggtgcggccccacatgagtgaccgctccctatgatgcccatgacagcgggggtggagcccccgcggtgtatattagtgaccctaattacgggttgctgcccgtcatcattatttacttctcttgtaagccgccgtctacggacaacgttctgtaaacactccacatatagtgaaggtttcatctgtctggtgcccgtggtttttcccttcgcattgaaggggtttccacgtaaatctatgtgtcgtgctctgtgatttattggttgatctacttcgtttttcctgtcgtttgtgctaacacatcttagcttgattttgcaacctttgtttatcaggtgatgattgggagcacgaggagatcttcactgatgatgatgaggttgtggatgttgatatagagcaaagacctgagttagctgatcctgaggctgctcctcctgaaattaaacaggtaaacaatatatgtgatgtgcctgctgcgttatgctgctgtatgctatcactagatctgctgatactgttgccttaaccaggatcaacaattcgccattcataagatgtgatatctgatttccatcataatactcacaggatgacaacgaggatgaactaggctgcagtgacaacttgagcaagtccggccaggagctaaagaggctgctccgccgagctgctggggaaaatgagtctgatgttgacgacaaaagcacatatgtaaggatcacaatcacaattcttcttagaactcaacatattcttttataggaggtgagaagtacacattagcaaacctaatgctatggaatagaactcatctttgtatctgtttcgtccgacaggaagatgacctaccctcgccaaaacctgctccaaaacttgtacccaaaagtgaaccagatgatagcagccctgctaaaccaacaccatcagggcacgctcagagcgctacaccttcatccaaatccactcaaaagaggaaatcagggggtggtgatgcaaatatttctaatggtgcaccctcgaaaaagataaagatggaacatgtacggcagtgtcttcattctgcacgctacacgtgaaatttgagttgcagacatattaagctgcacctgcaccaggttctttgctttaaaatttctctatttcttggcataataggaaaccggaacattggctgtcaaggatgaaaatccgtcttatttggaaccaacttcgaaagcatctctttcagcaagaagagaatcatcacccattacagaggaagaagtcaggacagtacttcgtgcaatttcacccacgacaagccaagatctggtgtcgaggttcaagactcgtctaataactcaagaggtaggcttcttcaaccttttgattacatgtttgttcttaatgcattaccataccttttaaaagttgtattacagtaattttttacttatttcattcagtaatccatggccttctattctcttattgctgcaggacaagaaagcatttttgggcattgtgacgaaaatatctcgcatacttaagcacaacggcggtaggtatatcgttcttcggcacgaacatatgtgaacagacaaattactctacctgtatagtgcaacagattctttaatgccacaaggcaacagcatgagtttttccccctagcttagagcttagggtttaggtattaggtatattgggtactggcaaccaagatgctcagcaagaggcctctgcagtttttctcccttataatgtacttcctgagagctgagaatgaaatgagaaaacgagaatggaatctatggtagattcttataatccaatcacaagattttg

SEQ ID NO: 4 - protein, 552 aa, Zea mays
MASNQTNQTLRSEHTAGGGISMGSVAPAELVLKAACERCGAASGLYATSCRHTTLCSDCGMTLALARGRCTVCSAPVTRLIREYDVRVDTSAEKEKTHYIGRFTTGLPPLSNERSAGNRWTLRKDVPHGQQLTGNMRDKHYSKMPWILEDETGEHQYQGQTEDPPATYYSLTQKGNDMIAVQVGSWYNFSKIAQYKQLTLEEAEEKMNRRRSSASGYERWMMKAATNGPDAFSSVVKRLDDVNRGATNGVHPKKGDRNENGNQSDKVDDKEEEGAARKNRLGLTMKDMGEDYEDSGNDIEFDLDDEIEKGDDWEHEEIFTDDDEVVDVDIEQRPELADPEAAPPEIKQDDNEDELGCSDNLSKSGQELKRLLRRAAGENESDVDDKSTYEDDLPSPKPAPKLVPKSEPDDSSPAKPTPSGHAQSATPSSKSTQKRKSGGGDANISNGAPSKKIKMEHETGTLAVKDENPSYLEPTSKASLSARRESSPITEEEVRTVLRAISPTTSQDLVSRFKTRLITQEDKKAFLGIVTKISRILKHNGGRYIVLRHEHM

SEQ ID NO: 5 - DNA, 2805 bp, Zea mays
  source 1..2805: mol_type = genomic DNA
gaggatagatactagtaggatatttagttttcttcaccatcaggaaaaaaaattggaaggaaatcagtccagtccagtcgaaatcagctactactatcatgcatgcttggttgcttgctcccctaccttgtgacaaagcaacgagcaccggaaaagggaaggaaaaaaaagagacgataataacctggactttgttcgttttgttgtttcaagaaaagttcatcacccacttctctccctgcttggacgacgacggtagatagatcacttgggaaggaatggactaatggaacagtgaaatagccatcgatgcggtccgtcggtctgctagctagctagtttaccacttaccaccaggacggatcaacggccaccaatggtggtcaccctcttcttgttttttgtcaggttgtcggtggtcactcgagcttgagcggcagcggggagccgggaccgccgcggtgacaggacgacccggcctgcggcggcggcggcggcggcggcatgactccagcgggcatctgcagaggcaagtgcagctgtgggggtggcggcggcggcggcggcgacgccatgcccgccatcgcctgctcctgcgccggcgaggcctcgaggcgcttggtggcgaggttgtgcttgttgttgtgcatccacaccttgagcacgcgccgcttcacgccgatctcctggcagaaggcctgcaccatgccgtcgtccagcttctggaggcgccaccccacgcgctccgcgaactccagcatgcgcgccttctgctccggggtgaacttggtgcggaaccgcttcctggacgacgacgacccgccgctgccgcgcgccgcgtccccgcagccgccgcggccgccctccatgtcgtcggactccgaggtgtgcatcatgttcagcggcatcacgtactggtggtggtggtgggcgtgccctggcggcggcggcagcgccggagggagcgcgcgcgccgacgcgtaggcggcgtaggggtcggccccggagatcaggagccccccgccgccgctcttgtggtgcgggtgcgccatggcgggggccaggaggcggcgggcggcacggtggccgtggccgtgcccgaggccgaggccgagaccgaggccgctgaagccttcggcggcgccgtacatgtcgtcgtcctcgtcgtccacctccttgcggtggaaattgcggtggcagccgcaggcggagcacttgagcgcctccaacgagccctcctcgccgcttggcatgaactcgccacacccgtcggtggcgttcccgccgatggccgccgcgtgattcttgaggcactcccggtacttcaccgtggagctgcccccgccgccgccggccccggctgccggaccgcgcttcttcgccgagctctccgtctcctccgccgcggcaggcggtggtggcagcggcggcgccggagactggccgttgttgctgccgtggtcacggtgcagctgcaggacatggccgccgacgccggctccgtagggcgacgcgacgttggcgtgcattgggatcgcgagctcgccctgcgtgccagatagatccatgcctcctggtcccgatccgtcgtcctcttctcggaggcgccgctccagagagaagatgcttcttgctggtgctgctgacgcttctgttctctgcagcggcacaatggaaaccatgatgagagagcgagcaagaaaaggccaatggcgcccagagagctcctagctcccctcactctcaagggtagatgcaacaaaattctcagtgcttcttgggactctcaacacaggcagaaggcactgaaggaaagagagagagactgtgagagagagagagagaggaggaggaggaggagggatcagagagaacaaaaagggtgctcctgcggtgaggtgcttggtgggggatcttgggaccttatcaacaagatactagttgcaattgcaaaccccaggagggaggttgtaagcaagctgctaaattttgaggccatagaataatgcatattgttatctgtgcttgctgccatgccctatactacatatctatcttgccagcttctttcttcttcttctgctactttcttgggctcttccccatgcaatgcaatgcaatgcgcacaagattacattacatacaagttggaagcaagggaggacggaaggaaggagaaagctactcggctatagtggaaggaagaaggatgaacagtgaggatgttgttcttaccaaaaggcagcaaggttactttgatgatctctcctggtcctggttactgctgggtccaagtatttactggttactgatatactgcacctactcttatactcttctgttttcctcccccactagatgtgtatatgtatgagctatatggacgagcctctgacacctccccttctctctctctctctaatcctaggctttacaacacaccacttcactcttaactccctctccttgaacgagacacggactaaattaaagctattgaggaggaagaggaggagctctcatgctgctgcagatgttttgtgtttaagcctagtgcagtgtttgtttttttgtttgtttgtgtgtgagtctcggaagccacaagaggttttccactagcaggctgctctgtgctctccctgtctgaacagcggaggaggaggagagagaaagggagatagagagagagaccaagagaccaaaagccaaccaaccttgttgcgtcaagggctaatcgagaggacaaaggagcaggggggcaagggcttcagcctttgcagcttccaccattgcagtgaggtgagg

SEQ ID NO: 6 - protein, 382 aa, Zea mays
MDLSGTQGELAIPMHANVASPYGAGVGGHVLQLHRDHGSNNGQSPAPPLPPPPAAAEETESSAKKRGPAAGAGGGGGSSTVKYRECLKNHAAAIGGNATDGCGEFMPSGEEGSLEALKCSACGCHRNFHRKEVDDEDDDMYGAAEGFSGLGLGLGLGHGHGHRAARRLLAPAMAHPHHKSGGGGLLISGADPYAAYASARALPPALPPPPGHAHHHHQYVMPLNMMHTSESDDMEGGRGGCGDAARGSGGSSSSRKRFRTKFTPEQKARMLEFAERVGWRLQKLDDGMVQAFCQEIGVKRRVLKVWMHNNKHNLATKRLEASPAQEQAMAGMASPPPPPPPPQLHLPLQMPAGVMPPPPPPPQAGSSCHRGGPGSPLPLKLE

SEQ ID NO: 7 - DNA, 1346 bp, Zea mays
  source 1..1346: mol_type = genomic DNA
tccaaacagcagcacgaagcacatatatataggactgggtacgcacagagatcgtcgtcaccaacttcgatctccaatccccagctagctaactatccaatggcgggccaagtgatggaagctcaggcgtgccggctgcagcctcccaccatggcgccgttcgcgccactacctcatcagtacaacaacacctgcaagcacctcaacacgacgatggccgccacttccggcaacaacaacagcaacggcagcaccgctaccgcggctggcggtgcggcggccgacggcatggccgcctacctccagcacctgcagcaggcgggcgccgaggcggcggccaagagcggcggcggcggcggcgcggcgcgcggggagcagtgcccgcggtgcgcgtcgcgcgacaccaagttctgctactacaacaactacaacacgtcccagccgcgccacttctgccgcgcctgccgacgctactggacgctgggcgggtccctccgcaacgtccccgtcggcgggtccacgcgcaagcgcccgcgcctggcgcaccaccaccagcacgccaggctcgctcctccggcgccgcacgtcttcggcctcacgccgatgatgcctcctcccttgcaaccgtcgtcgtcgacatcgtcgtcgcagggacagggacagggacagggacagagcggcggcctcctcggctcactgttcgcgctcggcgccgcgggcgcggggccgccgctgctcgaaggccgcggcgcagggtcgtcgttcgacttcgacctcgggctcggactcccgacgggggccttacacctgggcgcggccggacaaatgcaaggcctcgggctcatggggggaggaggtgcctccgcagccgggtcgtcgtccttcctctggcccgccgcggggctgctggtggacaacgacagcgtggacacgtggaagatgccaggcgccgctgcgggttcaatgtggccagacttctcttttccggcggcagcggcgccacaaaccgccggattgcttcatggcggaggccacctgatgtgagacgagacgccgcgcttccttcgacgtactaccccctagcgctagccctagattctagcgcatgcagcccggtgtccgggccggtttgcattgcatagcatacatagtagttggtctttttttcagcattgtattattggatcatgtgtacgtaattaagccgtgcttcagttttccttgactctgttaagctataaagggccctccaaatctgcagttttggggcgtttgtttcagaaaatcatattctagaattcgattctatcacaaaaaaaaacgtttatcagctcgttttttagaga

SEQ ID NO: 8 - protein, 314 aa, Zea mays
MAGQVMEAQACRLQPPTMAPFAPLPHQYNNTCKHLNTTMAATSGNNNSNGSTATAAGGAAADGMAAYLQHLQQAGAEAAAKSGGGGGAARGEQCPRCASRDTKFCYYNNYNTSQPRHFCRACRRYWTLGGSLRNVPVGGSTRKRPRLAHHHQHARLAPPAPHVFGLTPMMPPPLQPSSSTSSSQGQGQGQGQSGGLLGSLFALGAAGAGPPLLEGRGAGSSFDFDLGLGLPTGALHLGAAGQMQGLGLMGGGGASAAGSSSFLWPAAGLLVDNDSVDTWKMPGAAAGSMWPDFSFPAAAAPQTAGLLHGGGHLM

SEQ ID NO: 9 - DNA, 5573 bp, Zea mays
  source 1..5573: mol_type = genomic DNA
ttagagtattgacataatctaatctctatattgatacttgacaattgacataccaaacggtttataaacggaagcttcttaaaaggaaagcaatccataacgtctacagtagcaggcgttctttcaaagaggaccacactgtagtatagcatacaatgtttttttggcaaaggacaaacaaattgaacaaactaaatatcctatcatcctattttttagatctaacccttgctgcagaaagttgcaatttcagaagcagaatttaatgtccattgaacagacagagcatgttggtcactcgggcgatgaaggaatgctgggatcttaagggacattgtagctgatcaattatcagaaagaagtcatcaaggattgttgcacatcagtattccaagaaccaaaacatacttggagttggagcaaatctgatttgtaggtatacttcaccctatcaccctattaattcaatcaattgcattgcctttcagagatttaggttcatcagtgagtgtttacaagcaatgcatgcttacttcggtttatcctaattcctagaattgactttcccattgtcaagtacttttgtttgatcatcagttcctaacctgagcaacagatcgaagactaatcgatctaacagcagaactgggacttggtggcacatagtacattgcataggctaccaactcgggactcctggaattggtttcccctgtataaaaccataaaaccaagtgatccaggttgaattctgattaaacctcacaaagtggtgaggttcatactgttgtcatgacaaaccagtgtgaccagttgtcacccttgtagctggactaacagctaaatgaactgaaattttgattgtttggattcctgctagtttgtatacatgaaggcactcctgtagatctgcatcgcatgtcatcaagacccattctgaatcatcatccaagtacttcaaaaccaaggggtttgtgtctactatactcaaacgttttgctatctcatgcttcagttcttgaaaattgcactctgggttcagtctgaatctcactttttctgagccaaatgttgcctttactctcatatttgcatctgtgttctgcttgggattgggggacgacgggcttcgagaagaatgatgtccaccattagccttttcagtaacatcctgtagaagatcaattggagtctcattaactgagaaatgctcaaataatgaagtgttttctgtttgcagagtctgaatagagctgtctttcacaggattttctgacttcatctgatcaattataccctgttgtgcatgttttgttgcaccaccgctacatgagggacttgactcagaactgtggctgcacgaagagtgtgagtgtgaagttgatggcacattactgcttgacttgtggggttggtgcttgttgacgtcggtgagattgctctggttggttggaggaaccgtgacacttcctgacaaattgttgtcagatgatattgaggtgttcgtgatatccttgtacagggtgtttagctggaaagctgtctctgctccatgaaccgaatcaatgatctgttgaagctttcttagagagtggtctaccttcttgatctttcgcgatggccagcgggttataccatgctgcctgcaaattcttttgagggtcgtagggcacactgtgggagataaaaaaaagttagtatatttgcaaccagatcaaacacatatatatgcatgctgtcataaaagatgctatcacaatctgaagatgcaatgcacacatgataaggatgctgtctaccgccatgtctggaagaaatagttttacctatgttgactcacgaagctctaaatccatctattctaattctggaaaacatcatatcatgccactctgataagttagtttgtgggctttattttggaatgtgtgtcttcctgcgtgtatgccataattgccatcataataagttagtttgtgggcctaatggtgagatcatcatatagatcattacataatgtctatgcgtagtgttttgttttatccggttgctttttaactgtaagcaatgaatgagactaatcaatccagttctaggctatgtacactgtttgctcaatcaactagaaagtagaaaccgtaaggtcgatctgctcactgaatttttatttattatttgttatgtcagttcttgaaagttgactttatgctgtgctgtacttgctttttgtctcttcatgttcatgtgctttctatatacaggaaagtggactagtggtatactatacatggaaaggaaacacagatcacggtaagggtgataagaatgttcatcttctgcctatgggatgcatgtgatggttgactctctgatggaaagtaacactatattattgcttaatcagattagcatgctggctgcattagttgtacctctataattaaatgactgatggtatctacagaagcagtaaactatcctttgctgaatcttgcatttatctgatgattcactcacctccaaggctccttgctgcatctttcaggctaccagcgaagtactgccgaagaatctgcaaactaacggtcttctcggcctttgtacgcctttttgccttgctaccttcagcagttgcctgaacaccagatagagatggaccctgccccccaggtgagaacattccatgcatccgcaatgcagctaactcggattcttgctgtgatgttcctgccagacttgtccaagaggcctctcctctacgatctgctgaattgcatccaaaggacaactcaactttgtccttcccatgaggagtaaaagagttcagctcgttcatttccagcaccgtttcgtcttccatctctctatctgtgaccacacgtagggtctggcaaacacttcgcatggtgcctgacaaggagtccagcactgacttctgctcctccagtgcttcgcaatctgttggcagaaagaattccagcacaaagtctgctgtcccagtgcgtgtgcaccgcaggcggatcgccactgcaccttttaacttgaagatcttagcatggtgagacaaggagtattccaatttagttgaggatccaatgtctggcaagaagcatggctggtttgttgtaaaggccttccctgcaaccccctccccgcgcagaaggtggtgctcggagcaggcctcatggaagccccgcatctcggcttcattcacaaagcatgcttcatcaatggtggagatgcagtaccggtagttctcgtcggaatgtcggctgcctcgtttcccttgttgagcacatgtgacccatgtctgagctaatggcagcttgtgggtcaagcaagctgctctcaggacctccagtatctctggtaaagcatctttgtaggaagcagtgctgaactggtggataatcaataggagttgccatgaagtcataagtgagaagatataagcattggtgttcgtgtctcaatatcttacacacttgctttatattctctcttttccaaattataagtcgctttgattttttttgttacataattttttccatgtgtctaaatataacatatatctagatacaaaagaggatctatgtactaaaagaaccaaagcgacttataatctggaacagagggagtagctgatctagggtaggtaaattgaaacgtgcatagttttttttttcttcacaaacatgcaattttaagtagatttcatatcatgcctacattaagttggaatgtagaggtcctgagattcatatttttggaaaaatgaattatttcccaaaattcaaagctgtgacacagtgagtgtagaatttcttttctgctcacctttatgcgtggaatgcttgaaacttctgtacttctgagattaactgcctgcaaagaccctgttcactattagtaaagcccggacctggaaacactattgactttctacagtaataattggcatgcaggtacttcaaggtggcatcagtggctagactgctgttttcctttcctgcactaagctggtgatttgatctcctaacctaacacacttgcgatatacttctctgaactttcaggggcagtgagattgaggaggtttcatacctggagagcactgcaaatggtgttgagctcagaggtgaagtttatcttctgccttgtcatgatcagctcgatgacgcccaggcagttgtagcttcccttctcgaacactggcagacccatggtgccgtggacatcgaggtattgtgcatcccttaccctgggatactcgtagctggtgaagtacctgatgtccggcgaccactcggggagcctgccgataaagaccctccctggcagccccggcgaatcccccgacttaacatcggccgagaactggtacttggtggacacctccctgaaccggaggaggctgtcggagcggtggtcgagcgtgaacggttgctcgctggtggtcagcaccagtttcccgtcatcgcggttgaccggcacccagagctgcacgaggacgtcgccgtcgctctgcgtatcccggatgtaggacagggcctggctgaaccgctcctccacggaggaggggctcgcgccacccgtctgaatccaccagctggcgttcgcggccggcgccgccgtcgtcactgtggtggctgtcgcgtcagccgagaagaaggctgagagtggaggaaacgctaccgagttgggcgagcgtgagaccacctcgagccagccgttgtcgccggacagtagctgctcaaggagatccaagtcgacgccccccgcgccttcggacgtggtgcgtggtgcggagatgctcggaagctgcgggtctcctccctcttccatgcagatgcaggtaactctcccttcagaacctgcttacacagttcatcacgaacgtactgcatcagtcatcagtggtagcgatgatcttggtaacgataacaacaacacttgggcatgcaaagccatagtgcttcacttctgttctgctggtccatgcacatcttgtgctaggaaaccatggggacatgggaatccttgcgagatttttgtgctgggcatgtgtgccttcggattgcagggtgtgggtggtaagcaagatcacatcattttcaatggaaagcaagcagcgaaagagatttgtagtaagagagtagcaagcagctcactgttaatcacacgcctggcgcctcccttcttgtcataaaaagggctcccgttctcctctcttcctcctgtggattagctaggtagcacggttctgctcagctcaactcaccgccggaaaaggctcagtgctccacccgaggagcatctaatggatcctgctgccgctgtgagacgaaaggacaacaagaaagttgcttggcctggcttcgcttggttttgcccttcgttttctggtgttgtcgtaacaacaaagactgtgggggccaccatggcaattttgtttttctttatttgttagaatctttccctctcaccgagggcccagttgcgccacgcaggaatccaaccatacgtccatacccgcttccatatataaagtcaacttgcaagggtgatta

SEQ ID NO: 10 - protein, 851 aa, Zea mays
MEEGGDPQLPSISAPRTTSEGAGGVDLDLLEQLLSGDNGWLEVVSRSPNSVAFPPLSAFFSADATATTVTTAAPAANASWWIQTGGASPSSVEERFSQALSYIRDTQSDGDVLVQLWVPVNRDDGKLVLTTSEQPFTLDHRSDSLLRFREVSTKYQFSADVKSGDSPGLPGRVFIGRLPEWSPDIRYFTSYEYPRVRDAQYLDVHGTMGLPVFEKGSYNCLGVIELIMTRQKINFTSELNTICSALQAVNLRSTEVSSIPRIKFSTASYKDALPEILEVLRAACLTHKLPLAQTWVTCAQQGKRGSRHSDENYRYCISTIDEACFVNEAEMRGFHEACSEHHLLRGEGVAGKAFTTNQPCFLPDIGSSTKLEYSLSHHAKIFKLKGAVAIRLRCTRTGTADFVLEFFLPTDCEALEEQKSVLDSLSGTMRSVCQTLRVVTDREMEDETVLEMNELNSFTPHGKDKVELSFGCNSADRRGEASWTSLAGTSQQESELAALRMHGMFSPGGQGPSLSGVQATAEGSKAKRRTKAEKTVSLQILRQYFAGSLKDAARSLGVCPTTLKRICRQHGITRWPSRKIKKVDHSLRKLQQIIDSVHGAETAFQLNTLYKDITNTSISSDNNLSGSVTVPPTNQSNLTDVNKHQPHKSSSNVPSTSHSHSSCSHSSESSPSCSGGATKHAQQGIIDQMKSENPVKDSSIQTLQTENTSLFEHFSVNETPIDLLQDVTEKANGGHHSSRSPSSPNPKQNTDANMRVKATFGSEKVRFRLNPECNFQELKHEIAKRLSIVDTNPLVLKYLDDDSEWVLMTCDADLQECLHVYKLAGIQTIKISVHLAVSPATRVTTGHTGLS

SEQ ID NO: 11 - DNA, 10383 bp, Zea mays
  source 1..10383: mol_type = genomic DNA
ttttttcaaaagacatagctgtaaaatcaaacggcttctattttccacacgggccaccgctgtttttccacacagctcacagcatatttttcaaaacggcaaatcaaccaaacatactccaggttacttgcattatctcaccatagataggagtgagttaataaacaaattagatggcacgcctcattgttcatgtgaggatcgcgcaaccgaacctaaacagcgaactgtcaggcgcagactacccacgtcacagaacaagaacggcagctctgaagtagcggcggtctacaggggtaacggggtactcagtactcttgacttcatctgatatatacacacgggcacatctagcccgacgatcttgacttcggtcaattttgtgcaagcttcagcaggctaccatgggccagtcagggtgcggcgcaggcaaagcttctttgcctttggtccatgttggctgttgatacgctccaggacctgcaaaaaccatttacagagaagcttttgtgagtgaaaaaaaaacacattgtctgaagggaaccgtcatttcccagttgatgaatatgcagcgcatcaaaatcggcagccagaaagatctactgtcttccgctacaaaatagtcgtagaattttcaccaagattgtcagggaaaagcatgtttctcaggttaatgtgttaaaggtaacaatcagaaccaagaactatctgaatttcagaaaaaaaaatgttttgctattttatctcactaaatcataaggatgccaagacagatatcgttgttcactcactggtgagatttttttttctatcctttttcttttaagaaatatcaactaatcgggataagtacaaatatagacatggtagcactaagcaaggttcaattgctagtgttagcattctatttctaacaagtaaacatcacgccatataaatcacatttatgtgaagccctcttcgtttaaaatatgaagtaattaattagataaaagataaaaaaatataacacatcaaattctcaatagagttaactcattactttactaaattatatcaagaagagatgcatgaaaagtgtatgcaacaatttcaaaagtttcaattgaatgtgtgtctaattagcaatggtacatttctttgatgtcaaactaattcacattcacataggatactgattgatatgtacctagtgctctggtaggcttgtggtgaaattgtgaaagatatgtcaatattagaattcaaatatgttttcttgtagttacgagaaaccatgtcatctggaagttttggtatcctaaacatctcatgcatagaatggaatgttgtattgaaacctatacctgtgttgaaacccttgactggacatgccaaggtttatcacataagaactgagattgatattcctagcattgggtcaggtccagaccttggcccatactaaactgatcggctttttgcaatttgtattcacatttgaccatatgattcatttgaaattgttttggcgaattaactcttaagatcccttagaggaaggcaatagtctccagataatggcagtatagcttttgaaatcaaggggggcctcctagaactgcaggctatggcttgcattctggagtcaagttgatggtttctgtgtatctgacacgcctagatactcaaattacatgaaacaggcacaataaaacaggatggaaacgagaatcaagtcgtcgcatttcaatgccattcctatttatgtgtatctggtagctgcatttgaatgtcatgacttttgaatgtgtttcgcttagatgctcaaagtacacaaaaaaatcatgcagaacaaaaccagatggaagaaaagaaatcaagtcatccctatttccatgtaatgaattaatttcatttttggatactaattcaatcaatcttttttttttcttataaacactgaacatagtggatgaatggactggttgatatcatgttttacgaacaaatctatggttcagaaagtttttgggtgcaaaaagagaaacttacaaaatcatgtaacaaaatcatatgggcgtgaaaatggagtaaggtcatcatgcattgatttagtgataggtacatgttggagtaaattctagtcgtatttttaaagaaaaaaaaaatcaaactcatgtccaaccaaacatcagaaggtatctatcatagggaactactagaaagagccccactaaaatgttgtgaggctagacccgtgaaagttccatggaaaggattaaaacagtcatagaaaattaaatagaaaacataaaaatttgttgcttaacagcagcatatcatcagcccctgcctctttccccaatattaaaaaaaacatgcaatagtgatcaggctcaggcagagtctaaatagaacttgctaaagcttctacagaaacgaaatttttgaacatcagtatttttccaaactgaaagatcattacctcatccttttgcttctttagcatttcaacctgcacaatgaaacataaaagttaacatagcctctttcgtttttatcaggagttaagaagaaacaaactatcatggatgaaatggaatttagatctgaaataataaaaatccctgacaaactgtttggattctttcatttcagaagaattggaatctacataaaggactaggttatttggcttcaaatttgacattatagtaattttgaaagtttagatatagcatgtcttaaatccatgagctgagagatagaactagactccattgatccccatgctctgtttctcctttgcaacttatacaactcttcggctcgctgacatgcaacaacacttggacggccagacagtccacaccccggacggtctgcgggtggcccggacggtccgtgattagattaattcaggctgagtccttatctcgtgcgtggttattctaataaacccgcaaaaaaaaattggatctcacctaggaacgggtctagacctctccttataagggtacaactgattgagagacacacaatccaattgaaccaatctatttaccttatttacctttatgcattaagtctagatgtagcatagccttagttatagttccctaatcacagtcttcacctatcttcggctctacgtcgtctagaggcgctttgggtggcctaccgatcctgaagcaaccctacgatctctcctcccctacagggtccctcccgggaggcaagatctaggttcccgagagctctttgcgtcatcgcagatagtccggtgacctatcgcagacggtccggtgacctacaaagccgaccccaaggtcctacacatcgcgtgctggccatagggtttgttagtatttggcccaaaagcgagccaactttttggtgactccgctcgggacgactgagtttagatctatcagatcagatctattaggtcaaatctattagatcggtcacgatggtcaattccaaagatcacatcgatgtttccctagacaatatccttaggttgattatagaggatttgtcagccgctgagcagaaggaatttgaagactacgtgaagaagctggatgaggagttccaacgacaacaaggtcggaagcgcgaggaggcaaagaggtggttccgctcgcacttcaggatagatcgcgacaagaaggttgtctaggagagagaagtgaagctcgcttccctgccggccttgctgcagcagccccctactgtaagtaactctgatgatacccaatcccttaggtattatataaaatcaacaggaagatcaattgaaacggtacctagatctacaaatagaaacgctcaaaaaacttagatcatatggggctactaccaaggtattaatgccaagcacatcggcagcaaatggaatttcttagtcccagccatattatggtatgctaatgaactcatatttggagcaatcattgccacctcatgcactatacgataggttggcactgagtatggccggacaatccatgtacaatcacggaccgtccggccctctcgcaaaccgtccggctccgtatgccagactgtacggtacaatgccgggcccgccgtgtggcgcacaaacggtaccaaacattatcagacaattcaaacataatttcggaccgtccgatcccatagcggaccgtccgacaccttacgccggacagtccagcatcacattggactgaccaaccacccagtttgaaccgatcgggtacgtctacacaggacagggtgctgcacaataatacattttttctacccacatcaacaacactactatctcccacctacggtacatcaaagccgcagtataccatcaccaccaccatcggtatgcaacgcgcagctgattctgggtatggctagacagtccgagtacaatctcaaaccacccagacacctcatggactgtccgacacctttcgtcggactctctggaggcacaccgtgacaccaagcatgacacgcggcatccagatagtactccacacgaccggatagcccagatctaaccacgaactgctcagccaaatcgtggaccgtccgacaccgtacgccggaccgttcgtagtcccgcaggactgtccgaccactctcctcggaccgtctgtttatacatacgcgactgaacagtccgtataccacaccagaccgctcgacaaaatcgtggaccgtccgacaccctacgtcggaccgttcgtagtcccacaggaccgtccgaccactcctgccaaaccgactgactatacatatgcgaccaaacagtatgtataccacacaagaccttccgaccaagtcgcggagcatccgacaccttatgccggaccgtccggatatgcatacgcaaaacaaagtgatgcacatcaggcacaattttcgtactcacctcaacggcactactccaccccacctgcagcacaccgtagtcacaacatgccaccacccacgcatgaggccaaatattttttagcccctagggagctaaaaaggttcatccaataccacacgacagtgggatgcggtactgataagcctcagaggcctataccgaaagtctcctttgactgtcctagtacaaaaggccgacataatgataaccaaccacctgagccccatgatgagcatagtgacggtagtatctcagatgacgagttgaaagagttacatgtcgttgagcttgtttagcctactaaggccaaactttctgcttgctcttccctgtagccggttcaaaagaattagcaaggagaagctaagttcacatttaatgttgctaagtgtgacaaagtatttaatgagttacttaagaacgcaacattagattgtcccacacaattcctccgatagaggaattgaaaaggcgtgcttattgtaaatggcatggctccttttctcataccaccaatgattgtaatatcttccgtcgccaatacaatcggctatatatgaaagtcggttgagctttcaggaaatctagactgaccagcaacctttccccatcaaccctttaaagtcggtgggcaagaaagtcttgggtcggccgggtgtggctgacaaaagcaaaggacgattaaaccggtccaaaaggtgacgtagcaaatgtcgcttgctcatctacctccagttatgtcatggtactttccacctgtctactcatcgccaatgttttgtcctgctcgcatgtgggaaagtacgacgatgaatctgtatcacatgcttagtctatttgcttattcgggctaaggggcacaatagttttgtcccttttgacatattgatagtaatggttgaatgagatgcaatcctaatggacctttgtgcattagagttctattaaatgactatactttttagttgagagagcttgtgacttgcatcgagctagggttcttatttcgataacaagatgatttgtgaaatctattgtttctcgaaagatgctggtagtacaggtgcttttggttcgtgacctccaccaaaaggcagggggtcatatgttggacaccatatttggcacctggatggtcgggcagtccagtcatgtggctcggacggtccacgattagattaattcaagctgagtccatatcttgtgcgtggttatcctaacaaactcgcaggaaactgttggatctcacctaggaagggtccaggcctccccctatatgtatgaaggggcacaactgattgagagacacacaacccaattgaaccaacctatctaccttatttacctttatgcattagttctagatgtagcgtagccttagttgtagttccctaatctcaatcttcacctctctttgactctacatcgtctaaaggaactttgggtgacctacccgtcccgaagcaaccctaccctacgatctctcctcccgatggggtccctccatggagacgaaatctaggtttactcgagagctctctaggtcattgcggatgatccgacgacctgtcatggacagtccggcaacctaccgcgaataatccggtgacccgcagagccaaccccaaggtcctgcacatcacgtgctggccctagggttcattagtatttgtcccaaaagcgagccaacagtagcacacaagtatctcccctaatgtggccaataacaacatataagtatgctatacatgtggccatggtttttaaagcagtaaggtggtccaaggcgttggagcaccgcctggacgaggcaggcgggcaaggcgcctaggagttagaccaccgcccagacgcctaggcgacgccttaagaacagagcatgtggccatttaacttgatctgtgcctaaattgtgattattggaattaatatcaattccaacggtccaatggagcataagagtttaagcttggattgcatatgcccctttattttcatgtttgcgcctttctctctggctgcatgtgagtggggtgttgaagtgtataagtggattttctatcttctcttaacaacttaagtttttggtttgaactggctagttcattcactctaacagtctaacatggtatcagagccagagatctcaagttcaaatcctggcagagactttatttgtgcctccacccctttatttccacatttgtgcctttatttctggctgcacgtgagtgggagtgttgaagtgtataagtggattgcctaccttctcctaacaacttaagtttttgggttgaactggttagtccctaaaccctctatgtgatcaacatgcgaaaaccatcaatcatctgctcgttgggtgtgtgtttgctaggcaagtttggtttcggcttctccaacttgttgggctacaggctttgacaccgcaccctagagacactaattttgaagtgtgctggagacagtgcaacatgatggtcagtgcgtagagcaagaagcgtctcaactctttggtcatgttaggtgcttgaactatttggaaacatcgtaataactgtgtgtttcagggacaggcacccaatatatcctctgcctttcatgttgctaaagaataatttttcatatggtgtgtagctggagccaagggccttgctgccctgcaagctgtggcctgtggctgtaggttgagctgatccttttaatggatccctttatgtatcttcagttttggccaccgtgaggtggctttgtaccctgttttagtccattttggactttttctcttaatacaatgatacacagttcccctgcgattcgagaaaaaatctagttagtccattcactctaacagggtatcagagccaaaggtctcgagtttgaatcttggtagatgttatatttgtgcctccacacctttatttccacgtttgcgtctttccctccggctgcacgtgagtggccggaggtgtggaagtgtataagtagattacctatcttctcatcatagcttaaagtttttgggttgaactgattaatccattcactctaacaaagctaagcagaactttcctttatttgtctcaggtcaatgaggtgaggtgatgacgttaagtgctacacaatggacaatacaactatgcgacactagcaattttatcaataacatcagatgcccaggtgctacatgtgacaattgtggtctgataatttgatgtagacaatatacatttaaaaatagacataaatgaaagggtaccctaaatgaaaataccattgtaacacacattaagttcatgaaagatgtgtgcccaaaatagcaagctacgagctcaggtctaagacaacatgcatcaatctgtatgtgcagtcaggagtcagcatcagttgctcaaggcagctagttcgatgcagaacatatgcaataagggccaggcatttggaagttcctactaattgtagcaacttatctgctgccacgccagcagcaagcctcattataaaatttaaaagtctctagcagctcaaatctctagaaaaaaggaacccatgtagagaaagtctaatatgaagagattatcaaaggaacacagactgagtacattcattgatgctgtaaacaaataatgtgcatacaaccaagaaataaaatctatttttttagtggcaacaacatatcttttaacaaaacatagcaattatacctgctttttctgcaactcctcattctggtccttgagttttgccacctcagcttccagctccattatgtacgcctacaatgttaggacgcatgtggttaagtgtctgtacactgggttatgattttagactcttatcagtaaattgaaccaacataacaattttctatcaactagagcaatccagtaaatttgagtgccgatggggtcaaagactatagcttatacagcagtacatgtgcagcatggaataacttacagatgtaaaaaaatcaatcaatctcttgtatttgtatcaaaaactaaccagaaaagaacacaaaaccatgcgtttatgattggaattgtaaacctctttaagcccaagtagctatccctatcatctcaatgaggtggttgaaatgatgaagtctgtgagactggaatgtgtaccaccatctcccaaattaacaaacttatccagtgtctcagcctttctgaatctgagatggtcaagctgaaccatggctcattcccagattcgagaggcatacctctttagatgaagataagagtattatacaatggcaacttgctatttgtagaggctacttagcaataaattaacatcgaagagctaaactggaagtggcagatcacatcaagtgaataaagtacaacataatcgagaggcataccaaatatagtcaccatggagttccatttgcgacaagtttttactataattaagccaaagatgcagtatggttaacggacagcatagcagatccactgaaagaagaaaggctccacctgcttcctcgcacgcgacctggcggccgactccctgttcttgatcatgcggcgctgcctcctctccaccaccttctcgacggtgggtcccttccgcatcctcagcgcagtgtcgaacggatacggcaccggcgacagcgacgacagatcacccgcctccacctttcccaacccgttgaacacgaccggcgtcgtcggagccgccaccgtcacccctcctccgaggccctgcccgacgaccccggccaccatcccgtttcccagctgcagggtctgcggcgcgaccgcattcccctgggagaaaagcccctgcgcgtgcggctgcagcaggagggtctggtgcccccccatgtcctcacgaaccacgccggcgcgtaccaggaactcctccagagtcatggaccccagcgtctgctgcgcctgcgcctgcgccggcagcggcgcgggcgcgggcgcgggctcgggctcgggcgcgggagcgggagcgggagccgcaacctgctgcgcgtcctcgccgctggtgaggctcacgatctcgcgccagacctcgtcgaccgtcttctggctgagtgtgcggggcagcgtgaaggagccctggcgctggatgggcgccccgtgctgctgctgctgctgctgctgcgcgtgcgcgtccacggatgcggccgccgtggccgtggccgtggccggggcggccgcagccatggcgttagactcctcggctgtccagatgttgcgcagcagctcgtccatgttcatggagccgaagtccttgctggcgccgccgagcgtggtctggaactcgtcgaacgtgagcgagtagaccgagccctgccgcgccagcgctggggccccgctccctgccggcatctccatcttccccgcctaccgccttcttctcctctcctctccgctccgctccgctctcaaatcctaccggaattcaattccgcgcctggactgctgctctgaaaaaccagaggcggatgc

SEQ ID NO: 12 - protein, 351 aa, Zea mays
MEMPAGSGAPALARQGSVYSLTFDEFQTTLGGASKDFGSMNMDELLRNIWTAEESNAMAAAAPATATATAAASVDAHAQQQQQQQHGAPIQRQGSFTLPRTLSQKTVDEVWREIVSLTSGEDAQQVAAPAPAPAPEPEPAPAPAPLPAQAQAQQTLGSMTLEEFLVRAGVVREDMGGHQTLLLQPHAQGLFSQGNAVAPQTLQLGNGMVAGVVGQGLGGGVTVAAPTTPVVFNGLGKVEAGDLSSLSPVPYPFDTALRMRKGPTVEKVVERRQRRMIKNRESAARSRARKQAYIMELEAEVAKLKDQNEELQKKQVEMLKKQKDEVLERINSQHGPKAKKLCLRRTLTGPW

SEQ ID NO: 13 - DNA, 20 bp, synthetic construct
  source 1..20: mol_type = other DNA
attctccagcagataactct

SEQ ID NO: 14 - DNA, 20 bp, synthetic construct
  source 1..20: mol_type = other DNA
acctgagttagctgatcctg

SEQ ID NO: 15 - DNA, 20 bp, synthetic construct
  source 1..20: mol_type = other DNA
accgaggccgctgaagcctt

SEQ ID NO: 16 - DNA, 20 bp, synthetic construct
  source 1..20: mol_type = other DNA
caacggcagcaccgctaccg

SEQ ID NO: 17 - DNA, 20 bp, synthetic construct
  source 1..20: mol_type = other DNA
gagtcaagtccctcatgtag

SEQ ID NO: 18 - DNA, 20 bp, synthetic construct
  source 1..20: mol_type = other DNA
cttcctcgcacgcgacctgg